(19) United States
(12) Patent Application Publication
(10) Pub. No.: US 2019/0244677 A1
(43) Pub. Date: Aug. 8, 2019

(54) Systems, Methods, and Gene Signatures for Predicting the Biological Status of an Individual

(71) Applicant: Philip Morris Products S.A., Neuchâtel (CH)

(72) Inventors: Carine POUSSIN, Neuchâtel (CH); Vincenzo BELCASTRO, Yverdon-les-Bains (CH); Florian MARTIN, Peseux (CH); Stephanie BOUE, Hauterive (CH); Manuel Claude PEITSCH, Peseux (CH)

(73) Assignee: Philip Morris Products S.A., Neuchâtel (CH)

(21) Appl. No.: 16/333,157

(22) PCT Filed: May 30, 2017
(86) PCT No.: PCT/EP2017/063073

§ 371 Date: Mar. 13, 2019

Related U.S. Application Data
(60) Provisional application No. 62/394,551, filed on Sep. 14, 2016.

(51) Int. Cl. G16B 20/00; C12Q 1/6876; G16B 40/00; G16H 70/60

(52) U.S. Cl. CPC G16B 20/00; C12Q 1/6876; C12Q 2600/158; G16H 70/60; G16B 40/00

(57) ABSTRACT

Systems and methods for assessing a subject's sample to predict the subject's biological status, such as a smoker status. The computer-implemented method includes receiving, by a computer system including at least one hardware processor, a data set associated with the sample. The data set comprises quantitative expression data for a set of genes less than a whole genome, the set of genes comprising AHHR, CDKN1C, LRRN3, PID1, GPR15, SASH1, CLEC10A, LINC00599, P2RY6, DSC2, F2R, SEMA6B, and TLR5. The at least one hardware processor generates a score based on the quantitative expression data for the set of genes in the received data set, wherein the score is based on fewer than 40 genes and is indicative of a predicted smoking status of the subject.

### REFERENCE TO RELATED APPLICATIONS

This application claims priority under 35 U.S.C. § 119 to U.S. Provisional Patent Application No. 62/394,551, filed Sep. 14, 2016, which is herein incorporated by reference in its entirety. This application is related to PCT Application No. PCT/EP2014/077473, filed Dec. 11, 2014, and PCT Application No. PCT/EP2014/067276, filed Aug. 12, 2014, each of which is herein incorporated by reference in its entirety.

### BACKGROUND

Humans are constantly exposed to external toxicants (e.g., cigarette smoke, pesticides) that may trigger harmful molecular changes. Risk assessment in the context of 21st century toxicology relies on the elucidation of mechanisms of toxicity and the identification of markers of exposure response from high-throughput data. New technologies, such as whole genome microarrays, have been incorporated into toxicity testing to increase efficiency and to provide a more data-driven approach to exposure response assessment. Genome-scale inference of transcriptional gene regulation has become possible with the advent of high-throughput technologies such as microarrays and RNA sequencing, as they provide snapshots of the transcriptome under many tested experimental conditions.

The biomedical research community is generally interested in finding a robust signature for disease diagnosis. There is some evidence that molecular classification of diseases may be more accurate than morphological classification. However, sample acquisition from the primary site of exposure (e.g., the airways in case of smoke or air pollutant exposure) is usually invasive and is therefore not convenient for exposure assessment and monitoring. As a minimally invasive alternative, peripheral blood sampling can be employed in the general population to establish systemic biomarkers. Blood is complex to analyze due to the many different cell sub-populations it contains. However, it is a highly relevant tissue to investigate marker identification because blood circulates in all organs that are more directly exposed to toxicants and it is easily accessible. Moreover, molecular response to smoke exposure can be detected even when no histological abnormalities are visible.

### SUMMARY

Computational systems and methods are provided for using a crowd-sourcing method to identify a robust blood-based gene signature that can be used to predict a smoker status of an individual. The gene signatures described herein are capable of accurately predicting a smoker status of an individual by being able to distinguish between subjects who currently smoke from those who have never smoked.

In certain aspects, the systems and methods of the present disclosure provide a computer-implemented method for assessing a sample obtained from a subject. The computer-implemented method includes receiving, by a computer system including at least one hardware processor, a data set associated with the sample. The data set comprises quantitative expression data for a set of genes less than a whole genome, the set of genes comprising AHHR, CDKN1C, LRRN3, PID1, GPR15, SASH1, CLEC10A, LINC00599, P2RY6, DSC2, F2R, SEMA6B, and TLR5. The at least one hardware processor generates a score based on the quantitative expression data for the set of genes in the received data set, wherein the score is based on fewer than 40 genes and is indicative of a predicted smoking status of the subject.

In certain implementations, the set of genes further comprises AK8, FSTL1, RGL1, and VSIG4. In certain implementations, the set of genes further comprises C15orf54, CTTNBP2, RANK1, GSE1, GUCY1A3, LOC200772, MARC2, MIR4697HG, and PTGFRN.

In certain implementations, the score is a result of a classification scheme applied to the data set, wherein the classification scheme is determined based on the quantitative expression data in the data set. In certain implementations, the computer-implemented method further comprises computing a fold-change value for each of AHHR, CDKN1C, LRRN3, PID1, GPR15, SASH1, CLEC10A, LINC00599, P2RY6, DSC2, F2R, SEMA6B, and TLR5. The computer-implemented method may further comprise determining that each fold-change value satisfies at least one criterion that requires that each respective computed fold-change value exceeds a predetermined threshold for at least two independent population data sets.

In certain implementations, the set of genes consists of AHHR, CDKN1C, LRRN3, PID1, GPR15, SASH1, CLEC10A, LINC00599, P2RY6, DSC2, F2R, SEMA6B, and TLR5.

In certain aspects, the systems and methods of the present disclosure provide a kit for predicting smoker status of an individual. The kit includes a set of reagents that detects expression levels of the genes in a gene signature having fewer than 40 genes, the gene signature comprising AHHR, CDKN1C, LRRN3, PID1, GPR15, SASH1, CLEC10A, LINC00599, P2RY6, DSC2, F2R, SEMA6B, and TLR5 in a test sample, and instructions for using said kit for predicting smoker status in the individual.

In certain implementations, the kit is used for assessing an effect of an alternative to a smoking product on an individual. The alternative to the smoking product may include a heated tobacco product. The effect of the alternative on the individual may be to classify the individual as a non-smoker. In certain implementations, the gene signature further comprises AK8, FSTL1, RGL1, and VSIG4. In certain implementations, the gene signature further comprises C15orf54, CTTNBP2, RANK1, GSE1, GUCY1A3, LOC200772, MARC2, MIR4697HG, and PTGFRN.

In certain aspects, the systems and methods of the present disclosure provide a computer-implemented method for assessing a sample obtained from a subject. The computer-implemented method comprises receiving, by a computer system including at least one hardware processor, a data set associated with the sample, the data set comprising quantitative expression data for a set of genes less than a whole genome, the set of genes comprising LRRN3, AHHR, CDKN1C, PID1, SASH1, GPR15, LINC00599, P2RY6, CLEC10A, SEMA6B, F2R, CTTNBP2, and GPR63. The at least one hardware processor generates a score based on the quantitative expression data for the set of genes in the received data set, wherein the score is based on fewer than 40 genes and is indicative of a predicted smoking status of the subject.

In certain implementations, the score is a result of a classification scheme applied to the data set, wherein the classification scheme is determined based on the quantitative expression data in the data set.

In certain implementations, the at least one hardware processor computes a fold-change value for each of LRRN3, AHHR, CDKN1C, PID1, SASH1, GPR15, LINC00599, P2RY6, CLEC10A, SEMA6B, F2R, CTTNBP2, and GPR63. The computer-implemented method may further comprise determining that each fold-change value satisfies at least one criterion that requires that each respective computed fold-change value exceeds a predetermined threshold for at least two independent population data sets.

In certain implementations, the set of genes consists of LRRN3, AHHR, CDKN1C, PID1, SASH1, GPR15, LINC00599, P2RY6, CLEC10A, SEMA6B, F2R, CTTNBP2, and GPR63.

In certain aspects, the systems and methods of the present disclosure provide a kit for predicting smoker status of an individual. The kit comprises a set of reagents that detects expression levels of the genes in a gene signature having fewer than 40 genes, the gene signature comprising LRRN3, AHHR, CDKN1C, PID1, SASH1, GPR15, LINC00599, P2RY6, CLEC10A, SEMA6B, F2R, CTTNBP2, and GPR63 in a test sample, and instructions for using said kit for predicting smoker status in the individual.

In certain implementations, the kit is used for assessing an effect of an alternative to a smoking product on an individual. The alternative to the smoking product may include a heated tobacco product. The effect of the alternative on the individual may be to classify the individual as a non-smoker.

In certain aspects, the systems and methods of the present disclosure provide a computer-implemented method for obtaining a gene signature for predicting a biological status. The computer-implemented method comprises providing, by a computer system including a communications port and at least one computer processor in communication with at least one non-transitory computer readable medium storing at least one electronic database comprising a training data set and a test data set, the training data set over a network to a plurality of user devices. The training data set includes a set of training samples and the test data set includes a set of test samples. Each training sample and each test sample includes gene expression data, and corresponds to a patient having a known biological status selected from a set of biological statuses. The computer-implemented method further comprises receiving, from the network, candidate gene signatures that are each generated by obtaining a classifier based on the training data set, wherein each candidate gene signature includes a set of genes that are determined to be discriminant between different biological statuses in the training data set. A score is assigned to each respective candidate gene signature based on a performance of the respective candidate gene signature in predicting the known biological status of the test samples. A subset (or a portion of the candidate gene signatures that may include the entire set of candidate gene signatures) of the candidate gene signatures are identified based on the assigned scores, and genes that were included in at least a threshold number of candidate gene signatures are identified in the subset. The identified genes are stored as the gene signature.

In certain implementations, the computer-implemented method further comprises providing a number representative of a maximum threshold number of genes allowed in each candidate gene signature to the plurality of user devices.

In certain implementations, the computer-implemented method further comprises providing a portion of the test data set over the network to the plurality of user devices, wherein the portion of the test data set includes the gene expression data for patients having known biological status, and does not include the known biological status of the patients. The computer-implemented method may further comprise receiving, for each candidate gene signature, a confidence level for each sample in the test data set. The confidence level may be a value that indicates a predicted likelihood that a sample in the test data set belongs to one of the biological statuses. The score may be based at least in part on the confidence levels. In particular, the score may be based at least in part on an area under the precision recall (AUPR) metric computed from the confidence levels and the known biological statuses of patients in the test data set.

In certain implementations, the score is based at least in part on whether the corresponding candidate gene signature provides a prediction that is consistent with the known biological statuses of patients in the test data set. Whether the corresponding candidate gene signature provides the prediction that is consistent with the known biological statuses of patients in the test data set may be determined using a Mathews correlation coefficient (MCC).

In certain implementations, the candidate gene signatures are ranked according to at least two different metrics, to obtain a first rank and a second rank for each candidate gene signature. The first rank and the second rank for each candidate gene signature may be averaged to obtain the score for each respective candidate gene signature.

In certain implementations, the set of biological statuses includes smoker statuses. The smoker statuses may include current smoker and non-smoker.

In certain implementations, the gene signature is less than a whole genome and comprises AHHR, CDKN1C, LRRN3, PID1, GPR15, SASH1, CLEC10A, LINC00599, P2RY6, DSC2, F2R, SEMA6B, and TLR5. In addition, the gene signature may further comprise AK8, FSTL1, RGL1, and VSIG4. In addition, the gene signature may further comprise C15orf54, CTTNBP2, RANK1, GSE1, GUCY1A3, LOC200772, MARC2, MIR4697HG, and PTGFRN. In addition, the gene signature may further comprise ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618. In some implementations, the gene signature may be limited to a threshold number of genes, such as 10, 15, 20, 25, 30, 35, 40, or any other suitable number of genes less than the number of genes in the whole genome.

In certain implementations, the gene signature is less than a whole genome and comprises LRRN3, AHHR, CDKN1C, PID1, SASH1, GPR15, LINC00599, P2RY6, CLEC10A, SEMA6B, F2R, CTTNBP2, and GPR63. In addition, the gene signature may further comprise DSC2, TLR5, RGL1, FSTL1, VSIG4, AK8, GUCY1A3, GSE1, MIR4697HG, PTGFRN, LOC200772, FANK1, C15orf54, MARC2, TPPP3, ZNF618, PTGFR, P2RY1, TMEM163, ST6GALNAC1, SH2D1B, CYP4F22, PF4, FUCA1, MB21D2, NLK, B3GALT2, ASGR2, NR4A1, and GUCY1B3. In some implementations, the gene signature may be limited to a threshold number of genes, such as 10, 15, 20, 25, 30, 35, 40, or any other suitable number of genes less than the number of genes in the whole genome.

In certain implementations, the gene signature is less than a whole genome and comprises AHHR, P2RY6, KLRG1, LRRN3, COX6B2, CTTNBP2, DSC2, F2R, GUCY1B3, MT2, NGFRAP1, REEP6, SASH1, and TBX21. In some implementations, the gene signature may be limited to a threshold number of genes, such as 10, 15, 20, 25, 30, 35, 40, or any other suitable number of genes less than the number of genes in the whole genome.

In certain aspects, the systems and methods of the present disclosure provide a computer-implemented method for assessing a sample obtained from a subject. The computer-implemented method comprises receiving, by a computer system including at least one hardware processor, a data set associated with the sample. The data set comprises quantitative expression data for a set of genes less than a whole genome, the set of genes comprising AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618. The at least one hardware processor generates a score based on the received data set, wherein the score is indicative of a predicted smoking status of the subject.

In certain implementations, the score is a result of a classification scheme applied to the data set, wherein the classification scheme is determined based on the quantitative expression data in the data set.

In certain implementations, the computer-implemented method further comprises computing a fold-change value for each of AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618. The computer-implemented method may further comprise determining that each fold-change value satisfies at least one criterion that requires that each respective computed fold-change value exceeds a predetermined threshold for at least two independent population data sets.

In certain implementations, the set of genes consists of AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618.

In certain aspects, the systems and methods of the present disclosure provide a kit for predicting smoker status of an individual. The kit comprises a set of reagents that detects expression levels of the genes in a gene signature in a test sample, the gene signature comprising AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618, and instructions for using said kit for predicting smoker status in the individual.

In certain implementations, the kit is used for assessing an effect of an alternative to a smoking product on an individual. The alternative to the smoking product may include a heated tobacco product. The effect of the alternative on the individual may be to classify the individual as a non-smoker.

In certain aspects, the systems and methods of the present disclosure provide a computer-implemented method for assessing a sample obtained from a subject. The computer-implemented method comprises receiving, by a computer system including at least one hardware processor, a data set associated with the sample, the data set comprising quantitative expression data for a set of genes less than a whole genome, the set of genes comprising AHHR, P2RY6, KLRG1, LRRN3, COX6B2, CTTNBP2, DSC2, F2R, GUCY1B3, MT2, NGFRAP1, REEP6, SASH1, and TBX21. The at least one hardware processor generates a score based on the quantitative expression data for the set of genes in the received data set, wherein the score is based on fewer than 40 genes and is indicative of a predicted smoking status of the subject.

In certain implementations, the score is a result of a classification scheme applied to the data set, wherein the classification scheme is determined based on the quantitative expression data in the data set.

In certain implementations, the computer-implemented method further comprises computing a fold-change value for each of AHHR, P2RY6, KLRG1, LRRN3, COX6B2, CTTNBP2, DSC2, F2R, GUCY1B3, MT2, NGFRAP1, REEP6, SASH1, and TBX21. The computer-implemented method may further comprise determining that each fold-change value satisfies at least one criterion that requires that each respective computed fold-change value exceeds a predetermined threshold for at least two independent population data sets.

In certain implementations, the set of genes consists of AHHR, P2RY6, KLRG1, LRRN3, COX6B2, CTTNBP2, DSC2, F2R, GUCY1B3, MT2, NGFRAP1, REEP6, SASH1, and TBX21.

In certain aspects, the systems and methods of the present disclosure provide a kit for predicting smoker status of an individual. The kit comprises a set of reagents that detects expression levels of the genes in a gene signature in a test sample, the gene signature comprising AHHR, P2RY6, KLRG1, LRRN3, COX6B2, CTTNBP2, DSC2, F2R, GUCY1B3, MT2, NGFRAP1, REEP6, SASH1, and TBX21, the gene signature comprising fewer than 40 genes, and instructions for using said kit for predicting smoker status in the individual.

In certain implementations, the kit is used for assessing an effect of an alternative to a smoking product on an individual. The alternative to the smoking product may include a heated tobacco product. The effect of the alternative on the individual may be to classify the individual as a non-smoker.

### BRIEF DESCRIPTION OF THE DRAWINGS

Further features of the disclosure, its nature and various advantages, will be apparent upon consideration of the following detailed description, taken in conjunction with the accompanying drawings, in which like reference characters refer to like parts throughout, and in which:

FIG. 1 is block diagram of a computerized system for performing identification of a gene signature using crowd sourcing.

FIG. 2 is a block diagram of an exemplary computing device which may be used to implement any of the components in any of the computerized systems described herein.

FIG. 3 is a flowchart of a process for using crowd-sourcing to identify a gene signature for predicting an individual's biological status.

FIGS. 4A and 4B are tables that indicate co-occurrence across different teams for human data (FIG. 4A) and species-independent data (FIG. 4B).

FIG. 5 is a flowchart of a process for assessing a score that is indicative of a predicted smoking status of a subject.

FIG. 6 is a table that summarizes sample groups/classes, sizes and characteristics for different studies.

FIG. 7A is a diagram that illustrates identifying chemical exposure response markers from human and mouse whole blood gene expression data, and leveraging these markers as a signature in computational models for predictive classification of new blood samples as part of exposed or non-exposed groups.

FIG. 7B is a diagram that illustrates developing robust and sparse human (sub-challenge 1, SC1) and species-independent (sub-challenge 2, SC2) blood-based gene signature classification models (i) to discriminate between smokers and non-current smokers (task1), and subsequently (ii) to classify non-current smokers as former and never smokers (task2).

FIG. 8 is a diagram that illustrates releasing a training data set, a test data set, and a verification data set of blood gene expression data.

FIG. 9A is a boxplot that shows clear separation between smokers and non-smokers.

FIG. 9B includes two boxplots that show no significant difference between 0 and 5 days cession for the smoking group, but significant decreases for the Cess and Switch groups compared with their respective baselines at 0 days.

FIG. 10 includes two tables that show the class prediction performance of the gene signature classification model for class prediction.

FIGS. 11A and 11B are boxplots that show blood sample class prediction by the participants for the test and verification data sets.

FIG. 12 includes boxplots that show crowd log odds ratios between day 0 and 5 in confinement for the verification data sets.

FIG. 13 is a boxplot that shows crowd log odds distribution split per group/class and time of exposure to pMRTP or a candidate MRTP, or after switching to a pMRTP or a candidate MRTP.

FIGS. 14 and 15 are plots of MCC and AUPR scores to evaluate the performance of all possible combinations of signatures of lengths 2 to 18 with ML-based class predictions.

### DETAILED DESCRIPTION

Described herein are computational systems and methods for identifying a robust gene signature that can be used to predict a biological status of an individual. In particular, a biological status may correspond to the smoking exposure response status of the individual. The gene signatures described herein are capable of distinguishing between subjects who currently smoke from those who have never smoked or who have quit smoking. While the examples described herein relate mainly to smoker status or smoking exposure response status, one of ordinary skill in the art will understand that the systems and methods of the present disclosure are applicable to using crowd sourcing approaches to identify gene signatures for predicting an individual's biological status, where the biological status may refer to smoking exposure response status, smoker status, disease status, physiological state, chemical exposure state, or any other suitable status or state of an individual that is associated with the individual's biological data.

As used herein, an individual's biological status may be representative of various molecular changes that may occur in diseases or in response to exposure to one or more toxicants, drugs, environmental changes (such as temperature, microgravity, pressure, and radiations, for example), or any suitable combination thereof. Criteria are defined for a predictive classification model and are used in the computational analysis for the development and training of the predictive classification model. Features that discriminate between classes are extracted and embedded into the classification model for class prediction. As used herein, a classifier includes discriminant features and rules that are used for class prediction.

The crowd sourcing approaches described herein may be used to identify robust gene signatures to predict the exposure status of an individual to one or more chemicals. The study described in relation to Example 1 below involves an exemplary illustration of one such crowd sourcing approach for identifying gene signatures for predicting an individual's exposure to smoke. The study in Example 1 described below identifies both gene lists for human blood-based smoking exposure response gene signatures that are obtained from the crowd (e.g., multiple challenge participants), as well as gene lists for species-independent blood-based smoking exposure response gene signatures that are obtained from the crowd. The gene signatures described herein may be applied to one or more classification models that may be applied to new human (human signature) or human and rodent (species-independent signature) blood gene expression sample data to predict whether or not individuals have been exposed to smoke. The systems and methods described herein may be extended to identify gene signatures and one or more classification models to predict whether or not individuals have been exposed to one or more chemicals. While the study described in relation to Example 1 below relates to identifying blood-based gene signatures, one of ordinary skill in the art will understand that the systems and methods of the present disclosure are applicable to using crowd sourcing approaches to identify gene signatures that are not based solely on blood. Instead, the present disclosure is applicable to identifying gene signatures based on tissues and other features, such as protein and methylation changes, for example.

The systems and methods of the present disclosure may be used to identify markers capable of predicting exposure to toxicants. Indeed, robust marker-based classification models applied on a new sample may enable (i) prediction of whether a subject has been exposed or not exposed to a chemical substance and (ii) allow for monitoring of the magnitude of exposure response over time during product testing or withdrawal.

As used herein, a “robust” gene signature is one that maintains a strong performance across studies, laboratories, sample origins, and other demographic factors. Importantly, a robust signature should be detectable even in a set of population data that includes large individual variations. Robustness across data sets should also be properly validated in order to avoid over-optimistic reporting of the signature's performance.

Systems biology aims to create a detailed understanding of the mechanisms by which biological systems respond or adapt to external stimuli (e.g. drugs, nutrition and temperature) and genetic modifications (e.g. mutations, epigenetic modifications). New mechanistic insights are gained through the analysis and integration of large amounts of molecular and functional data generated using cutting edge technologies such as omics or high content screening. When applied in the field of toxicology, the overall approach termed systems toxicology, enables to quantify biological system perturbations triggered by xenobiotics (e.g. pesticides, chemicals), elucidate toxic modes of action, and evaluate associated risks. Systems toxicology has the potential to extrapolate short-term observations to long-term outcomes and to translate the potential risks identified from experimental systems to humans, suggesting that its application could become a new standard for risk assessment and decision making. The analysis of systems toxicology data as well as extrapolation and translation for predictive toxicological outcomes and risk estimates require the development of advanced computational methodologies. To demonstrate improved performance and reliability of new computational approaches, researchers may benchmark their own techniques against state-of-the art methods but often fall into what is called the “self-assessment trap” resulting in biased evaluations. Furthermore, the deluge of data generated and analyzed in systems biology/toxicology renders the review of published results and conclusions tedious for referees. Although reviewers can in principle access raw data that have been stored in public repositories, it is often difficult to reproduce an entire analysis by themselves. Therefore, there is a clear need for independent and objective evaluation or verification of methods and data involving an external third-party. The systems and methods of the present disclosure address this need and provide for a crowd-sourcing approach that receives submissions from researchers, identifies the best performing techniques, and aggregates their outcomes to create a robust gene signature for predicting a biological status.

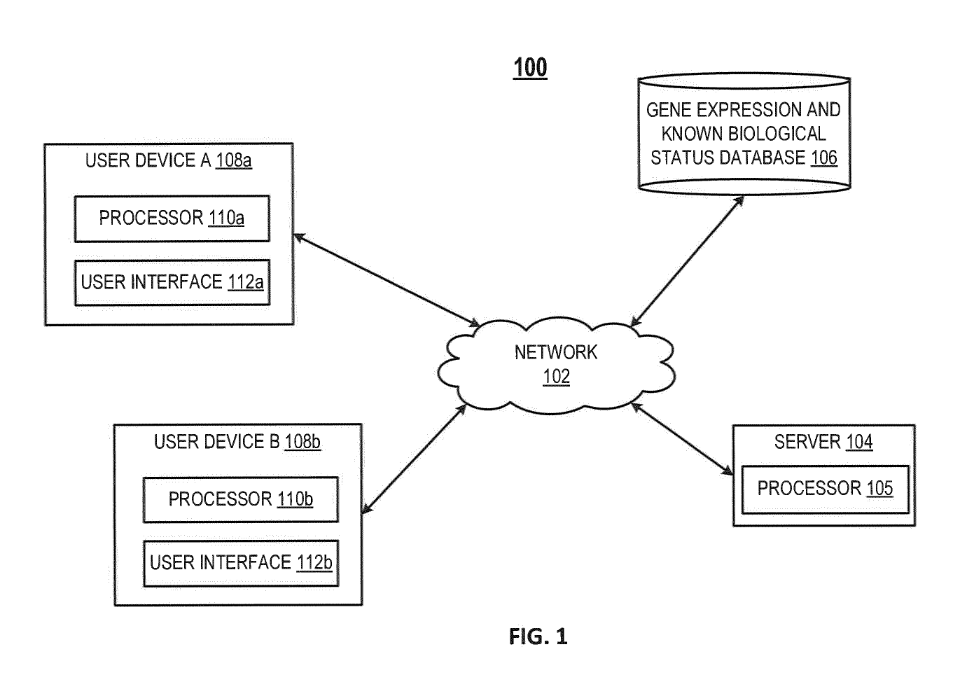

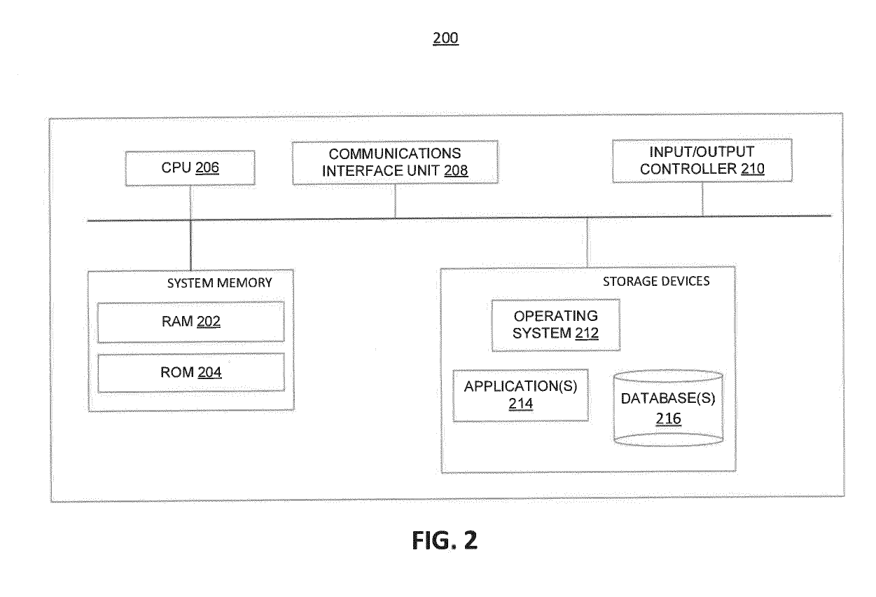

FIG. 1 depicts an example of a computer network and database structure that may be used to implement the systems and methods disclosed herein. FIG. 1 is a block diagram of a computerized system 100 for performing identification of a gene signature using crowd sourcing, according to an illustrative implementation. The system 100 includes a server 104 and two user devices 108a and 108b (generally, user device 108) connected over a computer network 102 to the server 104. The server 104 includes a processor 105, and each user device 108 includes a processor 110a or 110b and a user interface 112a or 112b. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that is currently being processed. An illustrative computing device 200, which may be used to implement any of the processors and servers described herein, is described in detail below with reference to FIG. 2. As used herein, “user interface” includes, without limitation, any suitable combination of one or more input devices (e.g., keypads, touch screens, trackballs, voice recognition systems, etc.) and/or one or more output devices (e.g., visual displays, speakers, tactile displays, printing devices, etc.). As used herein, “user device” includes, without limitation, any suitable combination of one or more devices configured with hardware, firmware, and software to carry out one or more computerized actions or techniques described herein. Examples of user devices include, without limitation, personal computers, laptops, and mobile devices (such as smartphones, tablet computers, etc.). Only one server, one database, and two user devices are shown in FIG. 1 to avoid complicating the drawing, but one of ordinary skill in the art will understand that the system 100 may support multiple servers and any number of databases or user devices.

The computerized system 100 may be used to leverage the wisdom of a crowd in identifying a gene signature for predicting an individual's biological status. As described above, scientists studying systems biology often fall into a self-assessment trap resulting in biased evaluations. The crowd-sourcing approach described herein helps to avoid these biases by designing a challenge, opening it to the scientific community (by making data on the gene expression and known biological status database 106 available to the user devices 108, for example), receiving submissions from independent scientists or groups (from user devices 108a and 108b, for example), and aggregating the best-performing results or predictions. To ensure broad participation, the challenge may aim to address questions related to scientific problems of common interests, such as identifying a blood-based gene signature for predicting an individual's biological status or smoker status.

The challenge makes certain data associated with blood sample data obtained from a group of individuals available to the scientific community. In particular, the gene expression and known biological status database 106 (generally, database 106) is a database that includes data representative of known biological statuses of a set of individuals and gene expression data (obtained from blood samples from the set of patients). Each individual in the set of individuals (whose blood sample data are stored in the database 106) may be randomly assigned as a training sample or a test sample. In some implementations, the assignment of individuals as training or test samples may not be completely random. In this case, one or more criteria may be used during the assignment, such as ensuring that similar numbers of individuals with different biological statuses are in each of the training and test data sets. In general, any suitable method may be used to assign the individuals as training or test samples, while ensuring that the distributions of biological statuses are somewhat similar in the training data set and the test data set.

Each training sample and test sample includes gene expression levels measured from the individual's blood sample as well as the individual's known biological status (e.g., the individual's known smoker status). The training samples make up a training data set, and the test samples make up a test data set. The entire training data set is provided from the database 106 to the user devices 108, while only a portion of the test data set is provided to the user devices 108. In particular, the measured gene expression levels from the test samples are provided to the user devices 108, but the known biological status corresponding to the test samples are kept hidden from the user devices 108.

Scientists at the user devices 108 may analyze the training samples to attempt to identify any dependencies, associations, or correlations between the measured gene expression levels and the biological statuses of the individuals in the training data set. The identified correlations may have the form of a candidate gene signature and a classifier. The candidate gene signature includes a list of genes that are differentially expressed for samples that are associated with different biological statuses (e.g., current smoker versus non-current smoker). A scientist may use any suitable computational technique to identify the candidate gene signature using any feature selection technique such as filter, wrapper, and embedded methods. Extracted features are combined in a classification model trained using a machine learning approach such as discriminant analysis, support vector machine, linear regression, logistic regression, decision tree, naive Bayes, k-nearest neighbors, K-means, random forest, or any other suitable technique. The classifier includes a decision rule or a mapping that uses the expression levels of the genes in the candidate gene signature to assign a sample to a class, which may refer to a predicted biological status of an individual. In this manner, each scientist at each user device 108 identifies a candidate gene signature and a classifier based on the training data set.

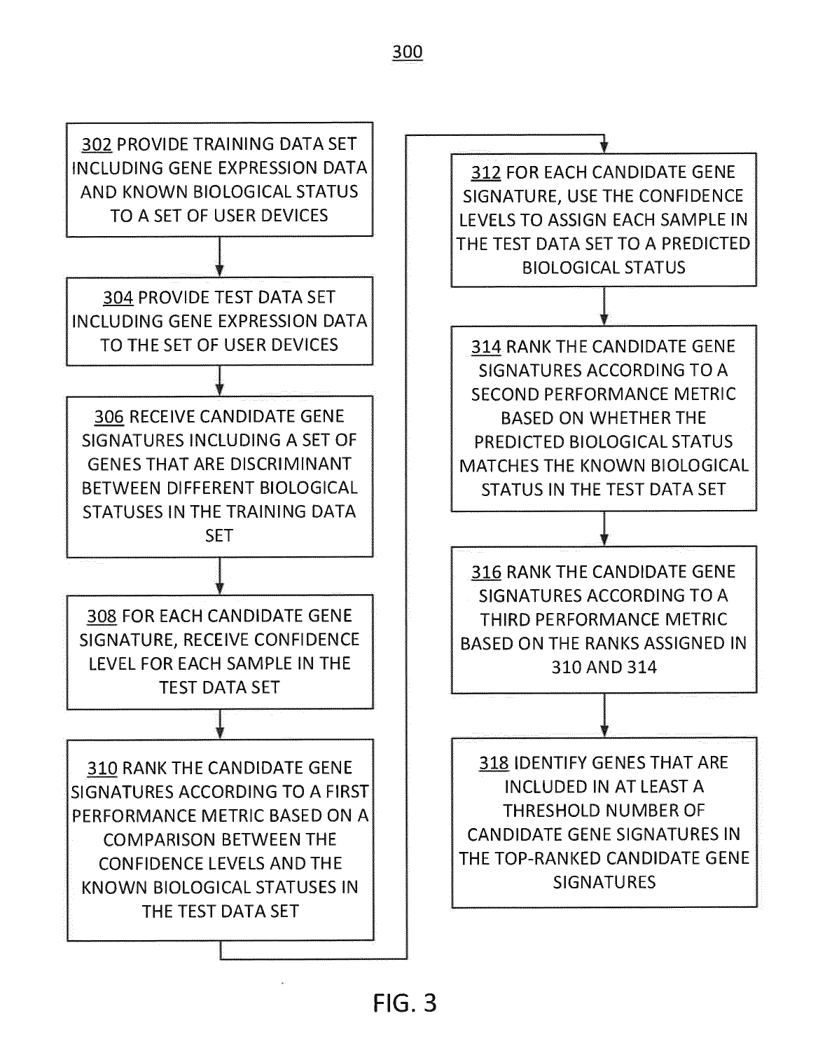

The scientists at the user devices 108 use their candidate gene signatures and classifiers to predict the biological statuses of the test samples in the test data set. The candidate gene signatures as well as a result obtained for each test sample are provided from the user devices 108 over the network 102 to the server 104. The submissions from the scientists may be anonymous. In one example, the result for each test sample includes a confidence level that corresponds to a likelihood or a probability that the corresponding test sample belongs in the predicted biological status. The confidence level is described in detail in relation to step 308 in FIG. 3. In another example, the result does not include a confidence level but rather only the predicted biological status for each test sample.

The server 104 may then identify the top performing candidate gene signatures by comparing the result obtained for each test sample with the known biological status for each test sample. In general, the best performing candidate gene signatures have results that closely match the known biological statuses. The server 104 then aggregates across the best performing candidate gene signature to obtain a robust gene signature that may be used to predict the biological status of an individual. This process is described in more detail in relation to steps 314, 316, and 318 in FIG. 3.

The components of the system 100 of FIG. 1 may be arranged, distributed, and combined in any of a number of ways. For example, a computerized system may be used that distributes the components of system 100 over multiple processing and storage devices connected via the network 102. Such an implementation may be appropriate for distributed computing over multiple communication systems including wireless and wired communication systems that share access to a common network resource. In some implementations, the system 100 is implemented in a cloud computing environment in which one or more of the components are provided by different processing and storage services connected via the Internet or other communications system. The server 104 may be, for example, one or more virtual servers instantiated in a cloud computing environment. In some implementations, the server 104 is combined with the database 106 into one component.

FIG. 3 is a flow chart of a method 300 for using crowd-sourcing to identify a gene signature for predicting an individual's biological status. The method 300 may be executed by the server 104 and includes the steps of providing a training data set including gene expression data and known biological status to a set of user devices (step 302), providing a test data set including gene expression data to the set of user devices (step 304), receiving candidate gene signatures including a set of genes that are determined to be discriminant between different biological statuses in the training data set (step 306), and for each candidate gene signature, receiving a confidence level for each sample in the test data set (step 308). The method 300 further includes ranking the candidate gene signatures according to a first performance metric based on a comparison between the confidence levels and the known biological statuses in the test data set (step 310), for each candidate gene signature, using the confidence levels to assign each sample in the test data set to a predicted biological status (step 312), ranking the candidate gene signatures according to a second performance metric based on whether the predicted biological status matches the known biological status in the test data set (step 314), ranking the candidate gene signatures according to a third performance metric based on the ranks assigned in steps 310 and 314 (step 316), and identifying genes that are included in at least a threshold number of candidate gene signatures in the top-ranked candidate gene signatures (step 318).

At step 302, a training data set including gene expression data and known biological statuses for a set of training samples are provided to a set of user devices 108. As is described in relation to FIG. 1, the training data set that is provided at step 302 includes training samples that include gene expression levels measured from an individual's blood sample as well as the known biological status of the individual. A scientist at the user device 108 receives the training data set and uses the training data set to train a classifier that provides a mapping between the measured gene expression levels and the known biological statuses. At step 304, a test data set including gene expression data is provided to the set of user devices 108. As is described in relation to FIG. 1, the test data set that is provided at step 304 includes test samples that only include the gene expression levels measured from an individual's blood sample, but does not include the known biological status of the individual. In other words, the known biological statuses of the test samples remain hidden from the scientists at the user devices 108.

At step 306, candidate gene signatures including a set of genes that are determined to be discriminant between different biological statuses in the training data set are received. Each scientist or team of scientists at the user devices 108 may provide a candidate gene signature to the server 104, where the scientist has determined that the combination of gene expression levels in the candidate gene signatures are discriminant for one or more criteria (such as the biological statuses or exposure response statuses of samples in the training data set). The user device over which the training data set is provided may be the same or different than the user device over which the scientist provides the candidate gene signature.

At step 308, for each candidate gene signature, a confidence level for each test sample in the test data set is received. The confidence level may be a value between zero and one, that represents a likelihood that the corresponding test sample belongs to a particular biological status. In one example, when there are two biological statuses (e.g., a first biological status and a second biological status), the confidence level may correspond to a value p, which refers to a likelihood that a particular test sample belongs to the first biological status. In this case, the value 1−p may refer to a likelihood that the particular test sample belongs to the second biological status. In general, multiple confidence levels may be provided for each test sample and for each candidate gene signature when there are more than two biological statuses.

At step 310, the server 104 ranks the candidate gene signatures (received at step 306) according to a first performance metric based on a comparison between the confidence levels (received at step 308) and the known biological statuses in the test data set. The ranking performed at step 310 causes each candidate gene signature to be assigned a first rank value.

One way to evaluate the performance of a candidate gene signature is to display the prediction results in a table that includes a predicted biological status in the rows and an actual biological status in the columns. Table 1 shown below is an example of one way to display the prediction results. The first row of the table indicates the number of individuals actually having a first biological status (e.g., true current smokers) and the number of individuals actually having a second biological status (e.g., non-current smokers) whose samples were predicted to be associated with the first biological status (e.g., predicted current smokers). The second row of the table indicates the number of individuals actually having the first biological status (e.g., true current smokers) and the number of individuals actually having the second biological status (e.g., non-current smokers) whose samples were predicted to be associated with the second biological status (e.g., predicted non-current smokers).

TABLE 1

| | Actual | Actual |
|---|---|---|
| | Biological | Biological |
| | status 1 | status 2 |
| Predicted Biological | True | False |
| status 1 | Positives | Positives |
| Predicted Biological | False | True |
| status 2 | Negatives | Negatives |

A perfect predictor will have all of the individuals actually having the first biological status accurately predicted as having the first biological status (true positives will be 100% and false negatives will be 0%), and all individuals actually having the second biological status will be accurately predicted as having the second biological status (true negatives will be 100% and false positives will be 0%). As described herein, individuals may be classified into multiple biological status, such as smoking statuses (e.g., current smoker, non-current smoker, former smoker, never smoker, etc.), but in general, one of ordinary skill in the art will understand that the systems and methods described herein are applicable to any classification scheme.

To evaluate the strength of a predictor (e.g., the classifier and the candidate gene signature), various metrics based on the values in the prediction results table may be used. In a first example, one metric is referred to herein as “sensitivity” or “recall”, which is the proportion of individuals who were accurately classified as a first biological status (e.g., current smoker) out of the set of individuals actually having the first biological status. In other words, the sensitivity (or recall) metric is equal to the number of true positives, divided by the sum of the true positives and the false negatives, or TP/(TP+FN). A sensitivity value of one indicates that every sample actually belonging to the first biological status was correctly predicted as belonging to the first biological status, but provides no information regarding how many other samples were predicted incorrectly to belong to the first biological status (FP).

In a second example, one metric is referred to herein as “specificity,” which is the proportion of individuals who were accurately classified as a second biological status (e.g., non-current smoker) out of the set of individuals actually having the second biological status. In other words, the specificity metric is equal to the number of true negatives, divided by the sum of the true negatives and the false positives, or TN/(TN+FP). A specificity value of one indicates that every sample actually belonging to the second biological status was correctly predicted as belonging to the second biological status, but provides no information regarding the number of samples having the first biological status that were incorrectly predicted as having the second biological status (FN).

In a third example, one metric is referred to herein as “precision,” which is the proportion of individuals who were accurately classified as a first biological status (e.g., current smoker) out of the set of individuals that were predicted to have the first biological status. In other words, the precision metric is equal to the number of true positives, divided by the sum of the true positives and the false positives, or TP/(TP+FP). A precision value of one indicates that every sample that was predicted to belong to a particular class (e.g., biological status) actually belongs to that class, but provides no information regarding the number of samples having the first biological status that were incorrectly predicted as having the second biological status (FN).

To be considered a strong predictor, high values in both sensitivity and specificity, in both sensitivity and precision, or in sensitivity, specificity, and precision, may be desirable. While the sensitivity, specificity, and precision metrics may be used herein for evaluating the performance of the candidate gene signatures, in general, any other metrics may also be used without departing from the scope of the present disclosure, such as the predictive value of a negative test (TN/(TN+FN)).

In an example, the first performance metric is related to an area under a curve (AUC) metric. In particular, the curve may correspond to a receiver operating characteristic (ROC) curve or a precision-recall (PR) curve. The axes of the ROC curve correspond to the sensitivity (or true positive rate: TP/(TP+FN)) and false positive rate (FP/(FP+TN)). The axes of the PR curve correspond to the sensitivity (TP/(TP+FN)) and precision (TP/(TP+FP)). In one example, the area under the PR curve (AUPR) is used as the first performance metric to obtain a first rank for a particular candidate gene signature. In another example, the area under the ROC curve is used as the first performance metric. While the PR curve and/or the ROC curve may be continuous, the present disclosure may use discrete values (as a threshold is varied), and one or more interpolation techniques may be used to compute the area under the curve.

At step 312, for each candidate gene signature, the server 104 uses the confidence levels to assign each sample in the test data set to a predicted biological status. In particular, for each submission from the scientists, each test sample is assigned to a predicted biological status based on the confidence levels in the submissions. In one example, when there are two biological statuses (a first biological status and a second biological status), the confidence level may have a value p that is a likelihood that the test sample belongs to the first biological status. Moreover, the value 1−p may correspond to a likelihood that the test sample belongs to the second biological status. In general, the scientists may submit multiple confidence levels when there are multiple biological statuses, and the predicted biological status for a particular candidate gene signature may correspond to the biological status having the highest confidence level.

At step 314, the server ranks the candidate gene signatures according to a second performance metric based on whether the predicted biological status (obtained at step 312) matches the known biological status in the test data set. The ranking performed at step 314 causes each candidate gene signature to be assigned a second rank value.

In another example, the second performance metric may correspond to a Mathews correlation coefficient (MCC) metric. The MCC metric combines all the true/false positive and negative rates, and thus provides a single valued fair metric. The MCC is a performance metric that may be used as a composite performance score. The MCC is a value between −1 and +1 and is essentially a correlation coefficient between the known and predicted binary classifications. The MCC may be computed using the following equation:

$$MCC = \frac{TP * TN - FP * FN}{\sqrt{( TP + FP ) * ( TP + FN ) * ( TN + FP ) * ( TN + FN )}}$$

where TP: true positive; FP: false positive; TN: true negative; FN: false negative. However, in general, any suitable technique for generating a composite performance metric based on a set of performance metrics may be used to assess the performance of a candidate gene signature and its corresponding predictions. An MCC value of +1 indicates that the model obtains perfect prediction, an MCC value of 0 indicates the model predictions perform no better than random, and an MCC value of −1 indicates the model predictions are perfectly inaccurate. MCC has an advantage of being able to be easily computed when the classifier function is coded in a way that only class predictions are available. In general, any metric that accounts for TP, FP, TN, and FN may be used as the second performance metric in accordance with the present disclosure.

At step 316, the server 104 ranks the candidate gene signatures according to a third performance metric based on the ranks assigned at steps 310 and 314. In particular, the first rank at step 310 is obtained based on a comparison between the raw confidence levels and the known biological statuses of the test samples, and the second rank at step 314 is obtained based on a comparison between the predicted biological statuses (assessed from the confidence levels) and the known biological statuses of the test samples. The first and second ranks may be averaged (or combined in some way) to obtain the third performance metric.

At step 318, the server 104 identifies a set of genes that are included in at least a threshold number (e.g., M) of candidate gene signatures in the N top-ranked candidate gene signatures. In an example, the N highest ranked candidate gene signatures according to the third performance metric are determined. Any gene that appears in at least M of these N candidate gene signatures are included in the genes identified at step 318, where M is less than N. In some implementations, (N,M)=(3,2), (4,3), (4,2), (5,4), (5,3), (5,2), (6,5), (6,4), (6,3), (6,2) or any other suitable combination of values for N and M, where N is an integer ranging from 2 to the total number of candidate gene signatures, and M is an integer ranging from 2 to N.

### Example 1—Introduction

An example study is described herein, in which a crowd sourcing method is used to obtain a robust gene signature for accurately predicting an individual's smoker status. One aim of the example study is to identify markers of chemical exposure response in blood by benchmarking computational methods for the identification of human and species-independent blood exposure response markers and models predictive of smoking and cessation status.

### Example 1—Study Population and Design

Whole blood samples are collected in PAXgene™ tubes during clinical and in vivo studies, or purchased from a Biobank repository. The sample groups/classes, sizes and characteristics for the different studies are summarized in the table shown in FIG. 6. Briefly, human blood samples are obtained from (i) a clinical case-control study conducted at the Queen Ann Street Medical Center (QASMC), London, UK and registered at ClinicalTrials.gov with the identifier NCT01780298; (ii) a biobank repository (BioServe Biotechnologies Ltd., Beltsville, Md., USA) (data set BLD-SMK-01). Samples from both these sources include smokers (S), former smokers (FS) and never smokers (NS) selected on well-defined inclusion criteria (FIG. 6); and (iii) clinical ZRHR-Reduced exposure (REX) C-03-EU and -04-JP studies corresponding to randomized, controlled, open-label, 3-arm parallel group, and single-center studies. The REX studies aim to demonstrate reductions in exposure to selected smoke constituents in smoking, healthy subjects switching to a candidate modified risk tobacco product (“MRTP”) or smoking abstinence/cessation (“Cess”) compared with continuing to use conventional cigarettes (smokers) for 5 days in confinement. In general, a MRTP may be a heated tobacco product. As used herein, a heated tobacco product includes products that generate an aerosol by heating tobacco or mixtures that include tobacco, without combusting or burning the tobacco during use. Mouse blood samples are obtained from two independent cigarette smoke (“CS”) inhalation studies conducted with female C57BL/6 and ApoE/mice for 7 and 8 months, respectively. Studies include mice randomized into five groups: Sham (exposed to air), 3R4F (exposed to CS from the reference cigarette 3R4F), prototype/candidate MRTPs (exposed to mainstream aerosol from a prototype/candidate MRTP at nicotine levels matched to those of 3R4F), smoking cessation (Cess), and switching to a prototype/candidate MRTP after 2-month exposure to 3R4F (Switch). Blood samples are collected at different time points.

### Example 1—Blood Transcriptomics Data Sets

Transcriptomics data sets are generated from whole blood samples collected in PAXgene™ tubes.

Data Generation from Human and Mouse Blood Samples

Total RNAs are isolated using a PAXgene Blood kit. The concentration and purity of the RNA samples are determined using a UV spectrophotometer (NanoDrop® 1000 or Nanodrop 8000; Thermo Fisher Scientific, Waltham, Mass., USA) by measuring the absorbance at 230, 260, and 280 nm. RNA integrity is further checked using an Agilent 2100 Bioanalyzer (Agilent Technologies, Santa Clara, Calif., USA). Only RNAs with an RNA integrity number greater than 6 are processed for further analysis.

Total RNAs are isolated from the samples in the PAXgene™ tubes according to the manufacturer's instructions (Qiagen). The quality of the extracted RNA, and cDNA quality following target preparation using a Ovation® Whole Blood Reagent and Ovation RNA Amplification System V2 (NuGEN, AC Leek, The Netherlands) and fragmentation (e.g., the size distribution of the final fragmented and biotinylated product is monitored using electropherograms) are checked using an Agilent 2100 Bioanalyzer (Santa Clara, Calif., USA). The quantity of cDNA is measured with a SpectraMax® 384Plus microplate reader (Molecular Devices, Sunnyvale, Calif., USA). The cDNA quality is determined by assessing the size of unfragmented cDNA using the Fragment analyzer (Advanced analytical, Ankeny, Iowa, USA). After fragmentation and labelling, the cDNA fragments are hybridized on a GeneChip® Human Genome U133 Plus 2.0 Array (Affymetrix) according to the manufacturer's guidelines. Raw transcriptomics data are obtained from microarray image analysis. For the QASMC study, blood transcriptomics data are produced by AROS Applied Biotechnology AS (Aarhus, Denmark).

Data Processing

Raw data (CEL files) from each data set are processed and normalized in the R environment (v3.1.2) using frozen Robust Microarray Analysis, fRMA v1.1. Frozen parameter vectors human (hgu133plus2frmavecs v1.3.0) are used by the frma and GNUSE functions. The custom brainarray cdf files for human (hgu133plus2hsentrezgcdf v16.0.0) are used for affymetrix probe-to-entrez gene ID mapping and resulting in one probe set for one gene relationship.

The data is passed through a quality check step, which removes all CEL files that did not pass one of the following cutoffs for the criteria described herein. First, for a given probe set j, the Normalized Unscaled Standard Error (NUSE) provides a measure of the precision of its expression estimate on a given array, i, relative to other arrays. Problematic arrays result in higher Standard Error (SE) than the median SE. Arrays are suspected to be of poor quality if either the NUSE median exceeds 1 or arrays have a large interquartile range (IQR). Arrays with NUSE values higher that 1.05 are removed. Second, the Relative Log Expression (RLE) compares for each array the level of intensity of a given probe relative to the median level of intensity for that probe across all j arrays. The array-specific distribution of RLE is used to determine if a particular array has predominately low- or high-expressed features. A median RLE not near zero indicates that the number of up-regulated genes does not approximately equal the number of down-regulated genes, and a large RLE IQR indicates that most of the genes are differentially expressed. An array with median RLE>0.1 (in absolute value) is considered an outlier and removed. Third, arrays with Median Absolute RLE (MARLE) greater than the median absolute deviation of all array data set MARLEs divided by the square root of 0.01 (or median(MARLE)/(1.4826*mad(MARLEs))>1/sqrt(0.01)) are considered to have bad quality chips and removed.

The custom Brainarray CDF files for mouse and human are used for Affymetrix probe to Entrez Gene ID mapping, resulting in one probe set for one gene relationship (HGU133Plus2_Hs_ENTREZG v16.0, Mouse4302_Mm_ENTREZG v16.0 respectively). The quality check excludes CEL files that do not pass minimum quality criteria. To facilitate data set handling, human and mouse gene expression data sets are provided with human gene symbols for both. Mouse genes are homologized to human genes using the NCBI/HCOP mapping file. In cases where mouse genes map to multiple human genes, only the human genes that match capitalized mouse genes are retained.

### Example 1—Challenge Overview

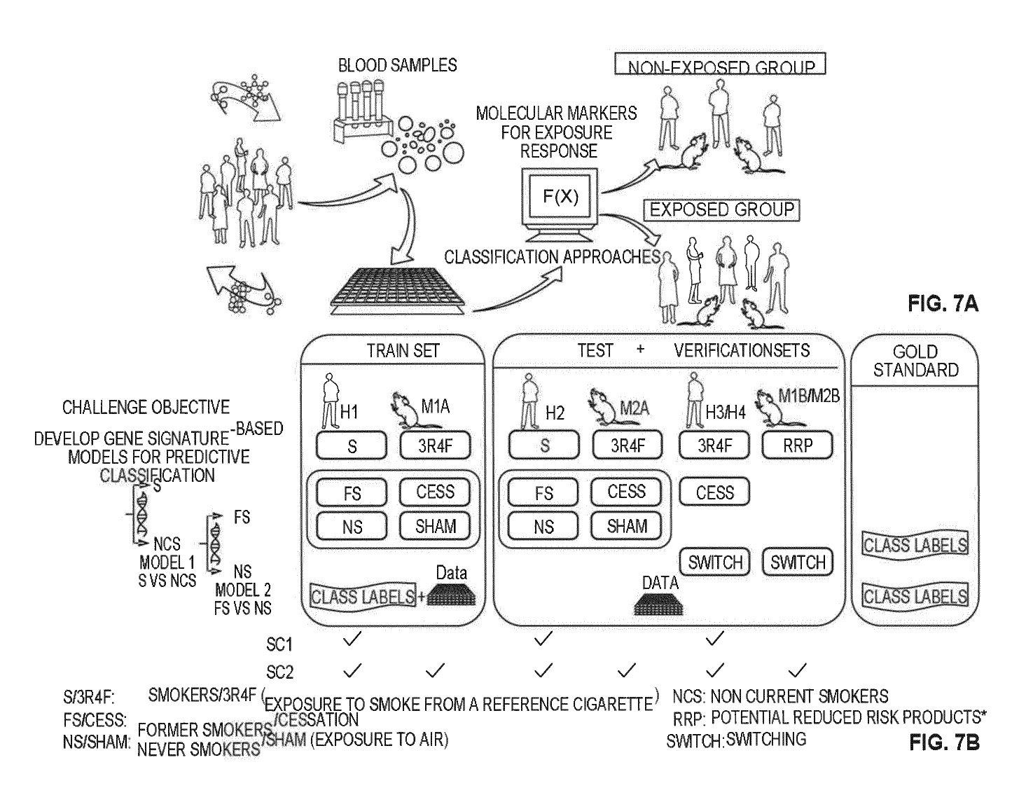

For the challenge, gene expression profiles from blood of smokers (S) and non-current smokers (NCS) subjects are provided to the scientific community, such as over the network 102 described in relation to FIG. 1. The set of gene expression profiles is evenly divided into a training set and a test set. The training data set (with full information on subject biological status: smoker, former smoker, never smoker class) is released before the test data set (with no information on subject biological status) is released. 135 registered scientists are grouped into 61 teams. 23 of the 61 teams provide submissions in line with the challenge rules, and 12 of the 23 teams provide eligible submissions. FIG. 7A shows an aim of the challenge is to identify chemical exposure response markers from human and mouse whole blood gene expression data, and leverage these markers as a signature in computational models for predictive classification of new blood samples as part of the exposed or non-exposed groups.

Data are obtained from blood samples collected in independent clinical and in vivo studies related to CS exposure and cessation in humans and rodents. The experimental groups also include individuals that are exposed to a prototype/candidate MRTP or switched to a prototype/candidate MRTP after being exposed to CS for a period of time. Participants are asked to develop models to predict smoking exposure based on a subject's gene expression profile generated from a blood sample. Specifically, participants are asked to solve two tasks: (1) identify smokers versus non-current smoker subjects, and (2) for each subject predicted as a non-current smoker, identify whether the subject is a former smokers (FS) or a never smoker (NS) subject. To be eligible for scoring, a team is required to submit predictions (e.g., a confidence level for each test sample) and a candidate gene signature (including a maximum of 40 genes) for both tasks. When the challenge is closed, anonymized predictions were scored according to a pipeline established with an external committee of experts. The best performers in the challenge achieved near perfect prediction to discriminate smokers from non-current smokers.

Challenge Goal and Rules

Participants are asked to develop robust and sparse human (sub-challenge 1, SC1) and species-independent (sub-challenge 2, SC2) blood-based gene signature classification models (i) to discriminate between smokers and non-current smokers (task1), and subsequently (ii) to classify non-current smokers as former and never smokers (task2, FIG. 7B). As a first constraint, predictive models are requested to be inductive (as opposed to transductive) with the ability to predict to which class a single new individual blood sample belonged without the need to retrain/refine the model or use a semi-supervised approach combining train and test data sets to predict sample class. As a second constraint, the signatures could include no more than 40 genes.

Data Released as Train, Test, and Verification Data Sets

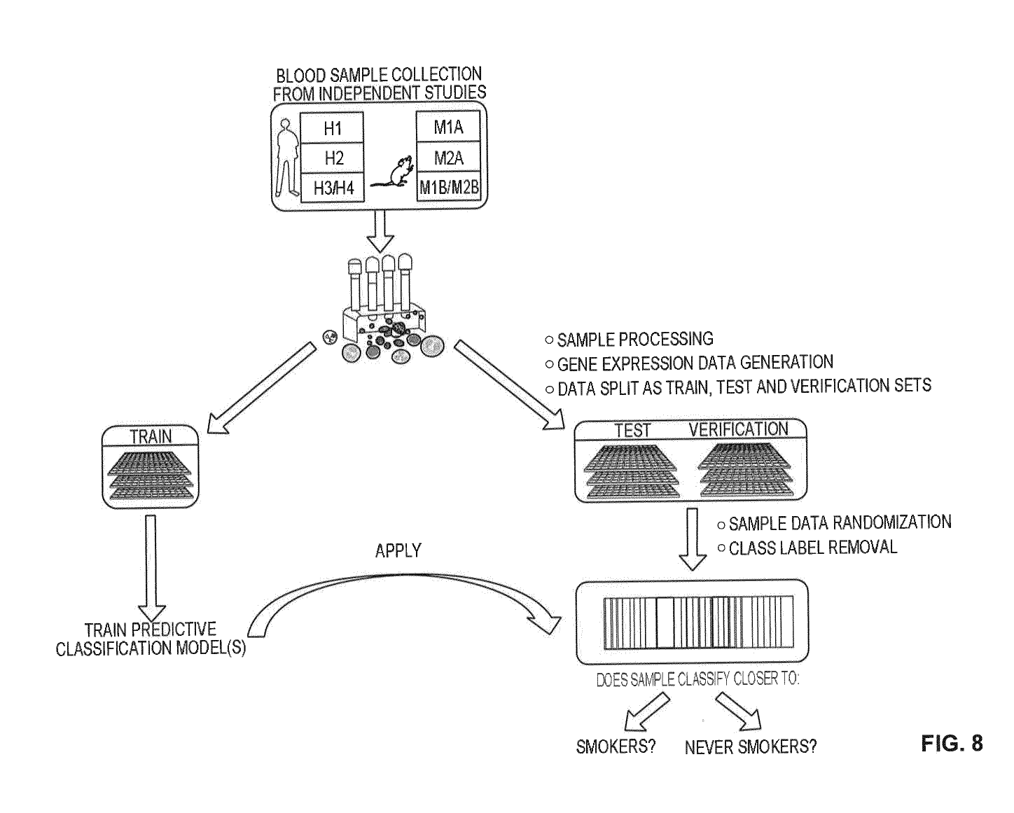

FIG. 8 shows a method of releasing the training data set, the test data set, and the verification data set of blood gene expression data. After blood sample processing and gene expression data generation, the data from independent studies are divided into training, test, and verification data sets. The data and class labels from the training data set are provided for the development and training of the blood-based gene signature classification models. Trained models are applied blindly on randomized test and verification gene expression data sets for class prediction of the blood samples.

Specifically, normalized gene expression data and class labels from the QASMC clinical (FIG. 7B, data set H1) and mouse C57BL/6 inhalation (FIG. 7B, data set M1a) studies are provided as training data sets. Human BLD-SMK-01 and mouse ApoE/data (FIG. 7B, data sets H2 and M2a, respectively) are used as test data sets. Data from the REX C-03-EU (FIG. 7B, data sets H3)/-04-JP (FIG. 7B, data sets H4) clinical studies, and mouse C57BL/6 (FIG. 7B, data sets M1b) and ApoE/(FIG. 7B, data sets M2b) inhalation studies are released as verification data sets. Sample data from test and verification sets are fully randomized and split into two class-balanced subsets that were sequentially released for class label prediction (FIG. 8). Samples from test data sets are used to score participants' predictions and assess team performance in each sub-challenge. The verification sets are used to evaluate whether participants predicted samples as closer to smokers or non-current smokers. Human data only, and human and mouse data are released for SC1 and SC2, respectively (FIG. 7B).

Predictive Gene Signature Classification Models

In order to avoid selection bias or to reduce the curse of dimensionality typically impacting the performance of whole array based gene signature, two public independent data sets are used to guide the filtering and gene selection. The highest fold-changes genes from the independent studies are jointly used by evaluating (for each N≥1) a linear discriminant model based on the genes in the intersection of the N highest fold-changes (in absolute value) of the two studies. The best N is chosen by 5-fold cross-validation (repeated 100 times) and leads to an 11-gene signature.

For the challenge, participants use various feature selection and machine learning approaches to identify discriminating features (genes) and classify samples. Random forest, partial least square discriminant analysis, linear discriminant analysis (LDA) and logistic regression are the classification methods used by the top three best performing teams in both sub-challenges. For each sample from the test and verification data sets, participants are requested to provide a confidence value P (between 0 and 1) that the sample belonged to class 1 (e.g. smokers), and a confidence value 1−P corresponded to the confidence value that the sample belongs to class 2 (e.g. non-current smokers). P and 1−P are requested to be unequal.

Scoring for Performance Assessment

Samples present in the test data set, and not in the verification data set, are used to assess team performance in each sub-challenge. Anonymized participants' class predictions are scored using Matthews correlation coefficient and area under the precision recall curve metrics. Overall team performance is based on the average rank computed across metrics and tasks (task 1: smokers vs non-current smokers; task 2: former smokers vs never smokers). Scoring results and final ranking are reviewed and approved by an external and independent Scoring Review panel of experts in the field. To evaluate team performance on the verification data set for this publication, the same scoring scheme is applied using smoker and former smoker (Cess) samples from the REX studies.

Post-Challenge Analysis

Confidence values corresponding to whether a blood sample belongs to the smoker or 3R4F groups are transformed as log odds (log(P/(1−P))). The distribution of the log odds for the individual top three teams (re-scored using the verification data set) or aggregated as the median across all qualified teams are visualized per class on boxplots. Paired (day 0 vs day 5 for longitudinal REX studies) and Welch t-tests were performed for key comparisons (i.e. all groups compared with their corresponding smoker/3R4F group). All statistical and graphic visualization is done using the R software v3.1.2.

### Example 1—Results

The case study in the present example reports results of an independent verification of methods and data in systems toxicology related to MRTP assessment. One aim of the study is to evaluate computational methods for the development of blood-based human and species-independent gene expression signature classification models with the ability to predict smoking exposure or cessation status (FIG. 7). Participants blindly applied their trained models on independent gene expression data sets that include smoker/3R4F and non-current smoker (former smoker/Cess and never smoker/Sham) data and data from mice that have been exposed to prototype/candidate MRTPs or human subjects and mice that have switched to a candidate MRTP after an exposure to conventional CS. For each sample, participants submit confidence values whether a sample belonged to the smoke-exposed or non-current smoke-exposed group.

Decreased Association of Samples from 5 Day-Cessation and Switching to Candidate MRTP Groups with the Smoker (S) Group Using a Human Smoking Exposure Gene Signature Classification Model

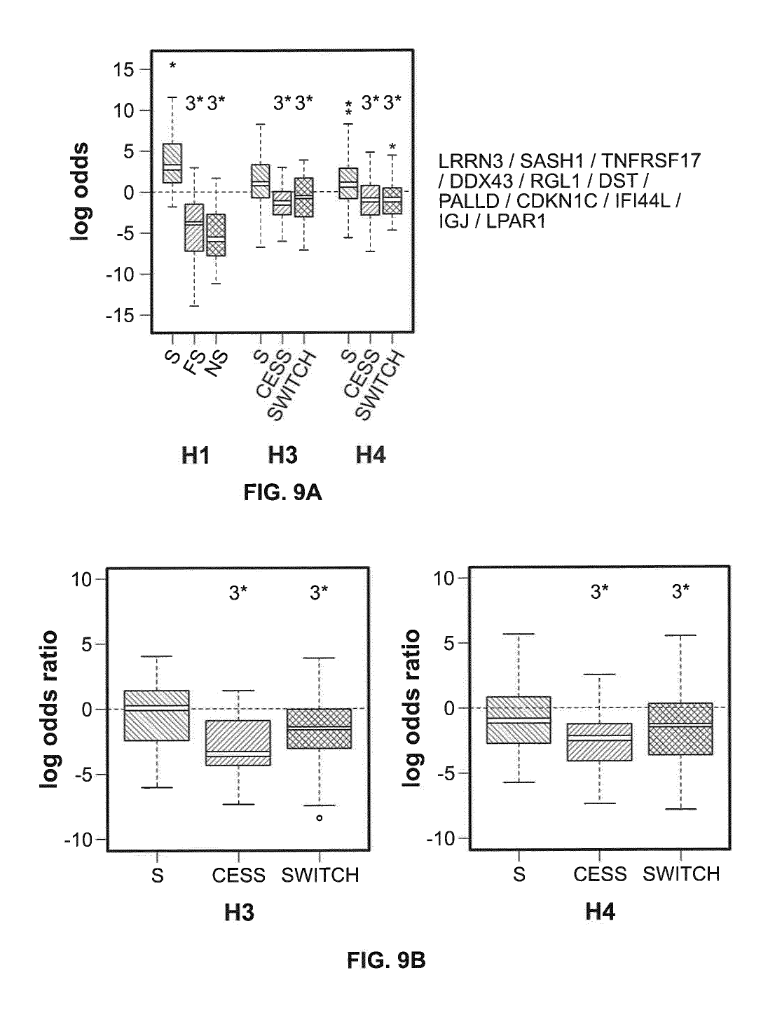

A human smoking exposure response gene signature classification model is trained on the QASMC data set that included smokers, former smokers and never smokers. The identified signature includes a set of 11 genes: LRRN3, SASH1, TNFRSF17, DDX43, RGL1, DST, PALLD, CDKN1C, IFI44L, IGJ, and LPAR1. To test the capacity of the signature to discriminate between smokers and non-current smokers, the model is applied on a test data set (BLD-SMK-01) and LDA scores with probabilities that a sample belonged to the smoker group are computed for each sample. The probabilities that a sample belongs to the smoker group (P) and the NCS group (1−P) are computed and transformed as log odds (P/(1−P)), to quantify the association of a sample with the smoker or non-current smoker group. The log odds distribution per group/class are visualized on boxplots (FIG. 9A, with a Welch t-test p-value 3*<0.001 vs S group). The median of log odds distribution for the smoker class is approximately +3.0, while the medians are approximately −3.8 and −5.8 for former and never smoker classes, respectively. The greater the median difference between smoker and non-current smoker classes, the more discriminative the gene signature classification model is. The boxplot shows a clear separation between smokers on one side and former and never smokers defined as non-current smokers on the other side (FIG. 9A).

The same model and procedure are applied directly on the verification data sets (REX C-03-EU and REX C-04-JP) to determine whether data from Switch or Cess subjects were classified closer to smokers or non-current smokers (FIG. 9A). In particular, Switch subjects are those who switched to a candidate MRTP, and Cess subjects are those who quit smoking for 5 days in confinement. After only 5 days of cessation or switching, the log odds related to these groups significantly decreases compared with the smoker group, whereas no difference is found between the Cess and Switch groups (FIG. 9A). No significant difference (log odds ratio) between 0 and 5 days is found for the smoking group, while significant decreases were observed for the Cess and Switch groups compared with their respective baselines at 0 days (FIG. 9B, Paired t-test p-value 3*<0.001).

Crowd Sourced Data Verification Confirmed the Prediction of Reduced Confidence that Blood Samples from 5 Day-Cessation and Switching to Candidate MRTP Groups Belong to the Smoker Group

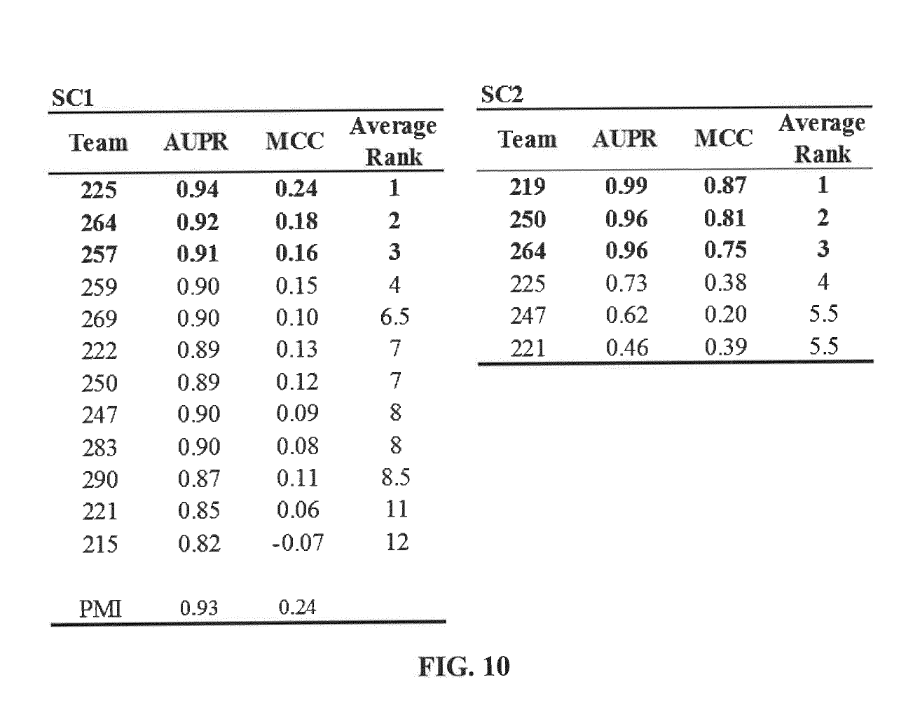

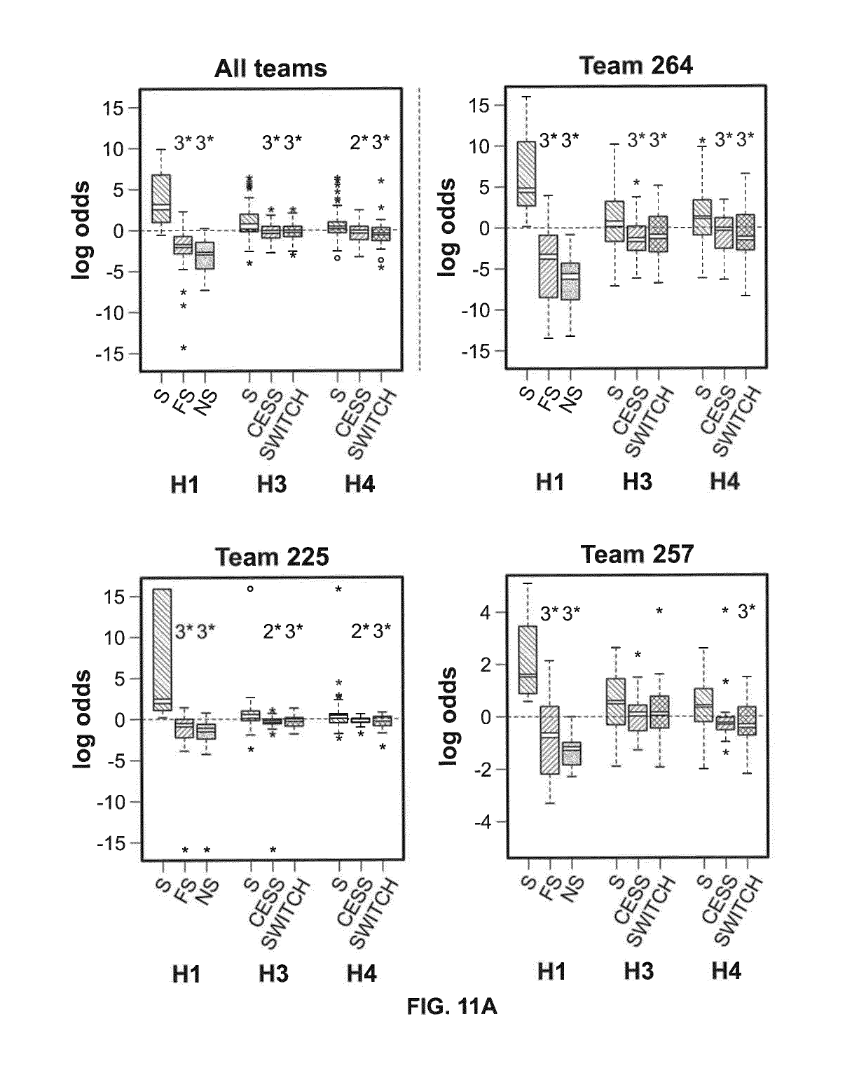

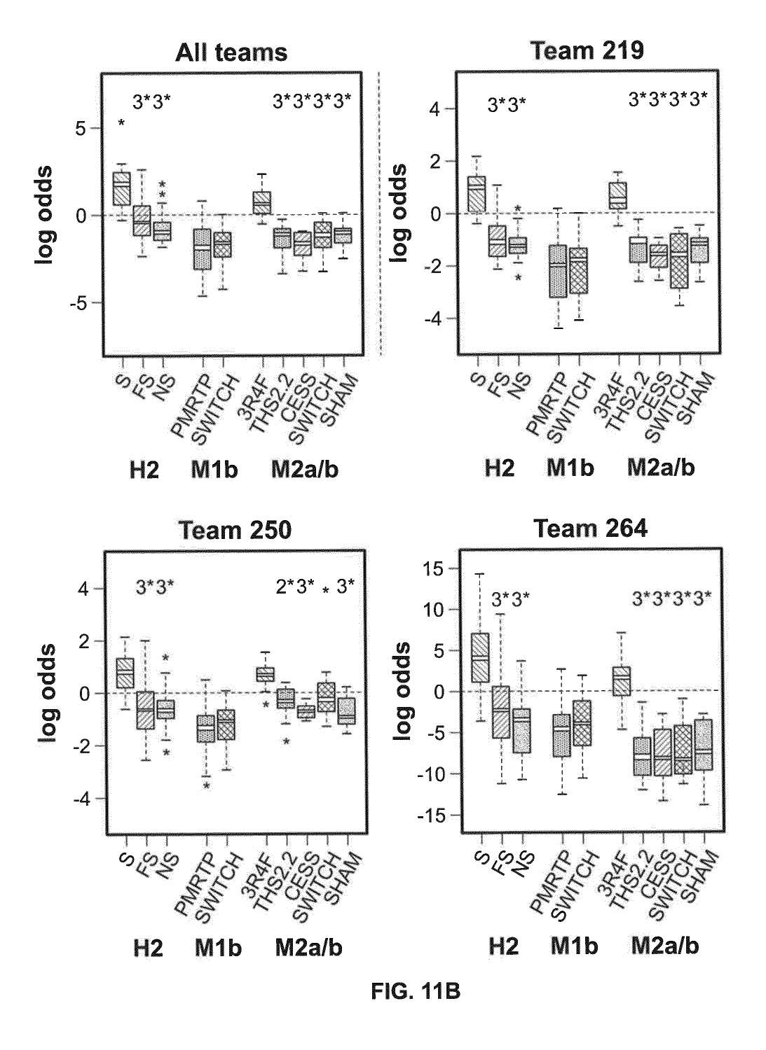

After training their human smoking exposure response gene signature classification model, participants applied their models on the randomized test and verification data sets and computed a confidence value (probability) for each subject that he/she belongs to the smoker group. After the challenge is closed, the scoring was performed on the test data set, which includes only smokers, former smokers and never smokers. The participants' prediction submissions are re-scored for the verification cohorts only, and teams 225, 264 and 257 are identified as the top three teams for SC1 (table shown in FIG. 10). The class prediction performance of the gene signature classification model for class prediction is assessed using the smoker and Cess (considered as former smokers for performance assessment) true class labels as a gold standard and the AUPR curve values are found to be at least 0.90 for the top three best performing teams (table shown in FIG. 10).

FIG. 11 shows human and mouse blood sample class prediction by the participants for the test and verification data sets. In particular, participants trained human (FIG. 11A) and species-independent (FIG. 11B) blood-based smoking exposure gene signature models to discriminate between smoke-exposed (S for human or 3R4F for mouse) and non-current smoke (NCS)-exposed (former smoker FS/Cess and never smoker NS/Sham) human subjects and mice. For each sample, participants are asked to provide a confidence value P that the sample belongs to the S/3R4F group, and a confidence value 1−P that the sample belongs to the NCS group. Confidence values are transformed as log odds (log(P/(1−P))) and are aggregated by computing the median of each sample across all 12 qualifying teams and displayed as distributions per class as boxplots (FIG. 11A). All the results show clear discrimination between smokers and non-current smokers (former and never smokers) for the test data set. For the verification data set, the observation of decreased association of samples from 5-day Cess and Switch groups with the smoker group obtained using the model was obviously confirmed by the individual or aggregated participants' predictions that produced similar results (FIG. 11A). The Welch t-test p-value is *<0.05, 2*<0.01, 3*<0.001 vs S/3R4F group. This confidence value drop toward the former/never class reflects that modifications in the signature gene expression occurred and are already detectable in blood cells after 5 days of cessation or switching to a candidate MRTP.

Crowd-Sourced Techniques Benchmarking Identified Best Performing Smoking Exposure Models for Blood Sample Class Prediction Irrespective of Human and Rodent Species

For SC2, participants are requested to develop a species-independent smoking exposure response gene signature model for class prediction that was directly applicable on both human and rodent data. The re-scoring of participants' prediction submissions using the verification data set identifies teams 219, 250 and 264 as the top three teams for SC2 (table in FIG. 10). For SC1, the confidence values obtained by the best performing teams or after aggregation of all team values are visualized as log odds distributions per class (FIG. 11B). A clear separation between cohorts exposed to CS/3R4F and those that are not exposed (never smoker/Sham and former smoker/Cess) is observable on the boxplots for both human and mouse, indicating that the models are able to classify blood samples irrespective of species (table shown in FIG. 10, FIG. 11B). When models are blindly applied on verification samples from two independent mouse in vivo studies, samples corresponding to the group exposed to a prototype MRTP (pMRTP) or a candidate MRTP have log odds values with similar levels to the Sham and never smokers control groups for the mouse and human data sets, respectively (FIG. 11B).

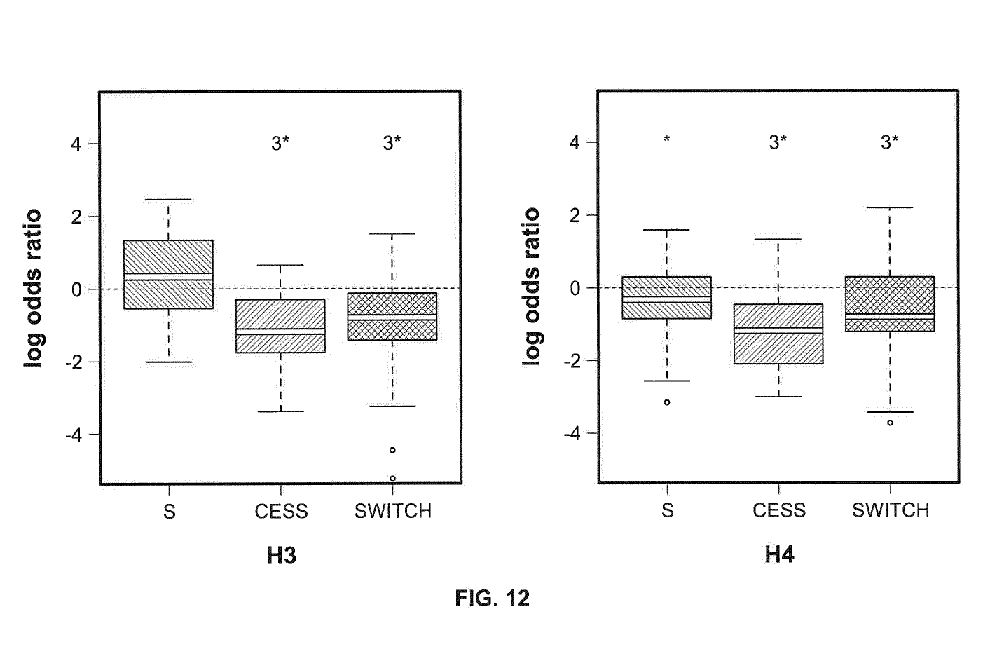

FIG. 12 shows crowd log odds ratios between day 0 and 5 in confinement for the verification data sets. Log odds ratios are significantly different between days 0 and 5 for the Cess and Switch groups, but, as expected, are not significantly different for the smoker group (paired t-test p-value 3*<0.001).

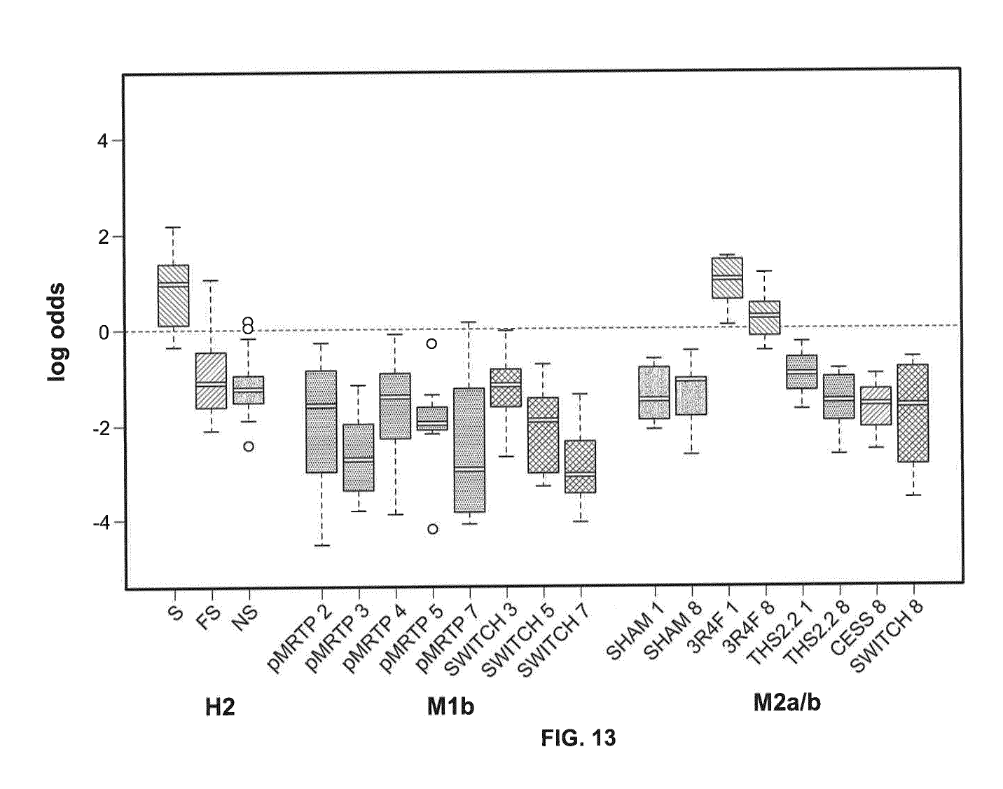

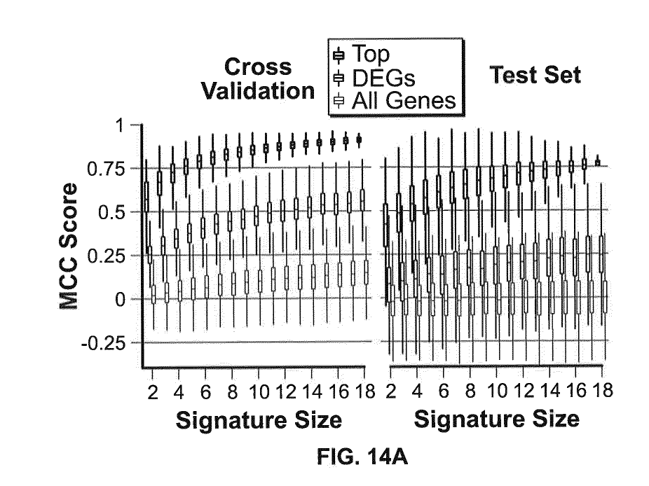

FIG. 13 shows crowd log odds distribution split per group/class and time of exposure to pMRTP or a candidate MRTP, or after switching to a pMRTP or a candidate MRTP. Specifically, after switching from 2-month CS exposure to pMRTP, a gradual decrease in log odds values is observed over time (e.g. Switch 3, Switch 5 and Switch 7 corresponding to 1, 3 and 4 months of exposure to pMRTP) when classes were split per time point, which is indicative of gradual gene expression changes occurring in blood cells over time.

Human and Species-Independent Response Markers in Blood Predictive of Smoking Exposure Status Show Commonalities and Included a Core Gene Subset that was Highly Consistent Across Teams

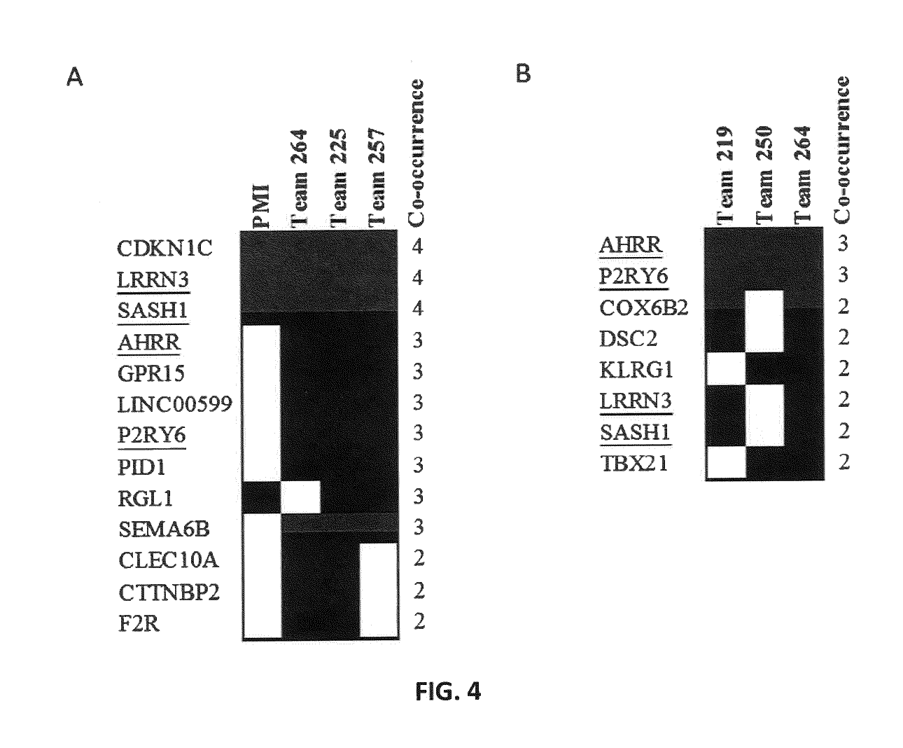

A smoking exposure core gene subset is identified by extracting genes with at least two co-occurrences across the top three team and PMI signatures (FIG. 4). Genes encoding cyclin dependent kinase inhibitor 1C (CDKN1C), leucine-rich repeat neuronal 3 (LRRN3) and SAM and SH3 domain containing 1 (SASH1) are the most frequently appearing genes in the human signatures (FIG. 4A), and genes encoding aryl-hydrocarbon receptor repressor (AHRR), pyrimidinergic receptor P2Y6 (P2RY6) have the highest co-occurrence in the species-independent signatures (FIG. 4B). A comparison between both core gene subsets reveals a common set of four genes encoding LRRN3, SASH1, AHRR and P2RY6 (FIG. 4).

### Example 1—Performance Analysis of all Gene Combinations from the Top Six Teams'

Human-Based Smoking Exposure Consensus Signature Impact of Gene Signature Length, Gene Expression Co-Linearity Level, and Classification Methods

Method

All possible combinations of genes from a consensus signature are considered. The extraction of an 18 gene-based human smoking exposure consensus signature is limited to the top six teams (instead of the 12 qualified teams) because of limitations imposed by the computer intensive calculation required for this analysis. The 18 gene-based consensus signature in blood, which included DSC2, FSTL1, GPR63, GSE1, GUCY1A3, RGL1, CTTNBP2, F2R, SEMA6B, CDKN1C, CLEC10A, GPR15, LINC00599, P2RY6, PID1, SASH1, AHRR, and LRRN3, is identified by selecting genes with at least two co-occurrences across the signatures of the top six teams. The impact of gene signature size and co-linearity level on classification performance is investigated. The analysis is conducted using five-fold cross-validated training (with 10 repeats) and test datasets from SC1, separately. The most widely applied machine learning (ML) methods in the challenge include Random Forest (RF), support vector machine with linear kernel (svmLinear), partial least squares discriminant analysis (PLS), naive Bayes (NB), k-Nearest Neighbor (kNN), linear discriminant analysis (LDA), and logistic regression (LR). All possible combinations of the 18 genes of length 2 to 18 (i.e. 262,125 gene sets) are generated. Applying each of the seven ML methods to each gene set leads to a total of 1,834,875 tested classification strategies. The level of co-linearity of genes within a gene set is reflected as the percentage of variance of the first principal component of the expression matrix restricted to that gene set. The performance of the 1,834,875 gene set-ML predictions (called “Top”) is evaluated by computing MCC and AUPR scores. The performance of these “Top” gene sets are compared with that of gene sets (2-18 genes) randomly selected among the differentially expressed genes (DEGs; false discovery rate, or FDR<=0.5) or all genes represented on the HG-U133_Plus_2 chip. The sampling process is repeated 1,000 times for each gene set size, leading to a total of 17,000 random “DEG” or “All genes” gene sets.

Results: Gene Set Combinations from an 18 Gene-Based Consensus Signature from the Top Six Teams are Informative and Outperform “DEGs” and “all Genes”-Derived Gene Sets for Smoking Exposure Status Class Prediction

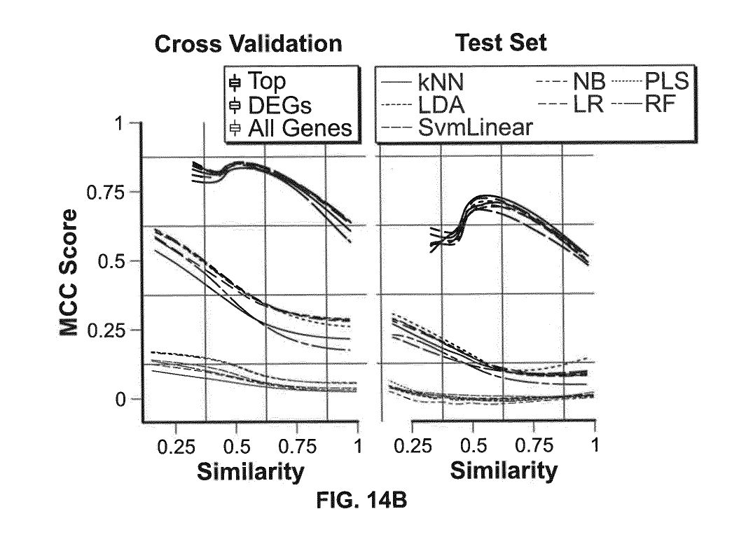

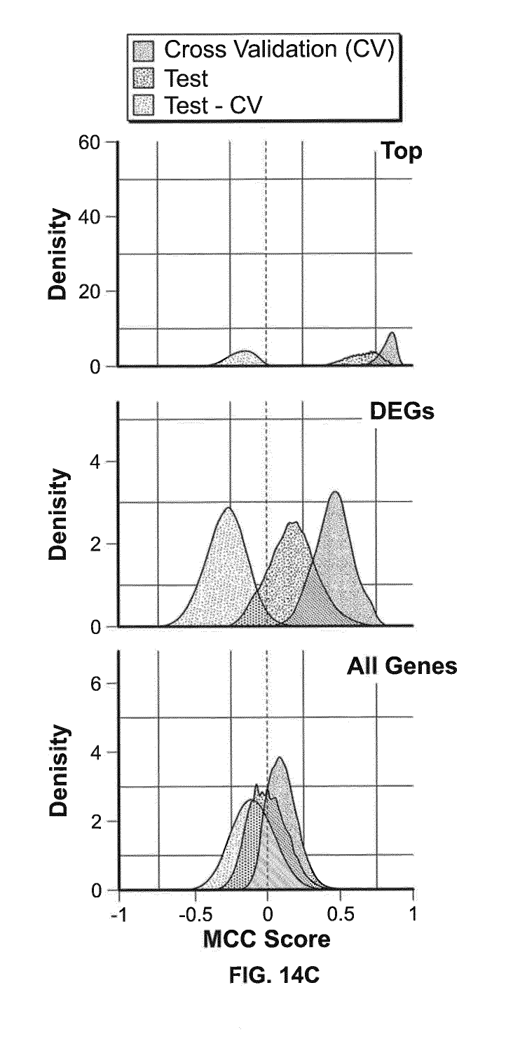

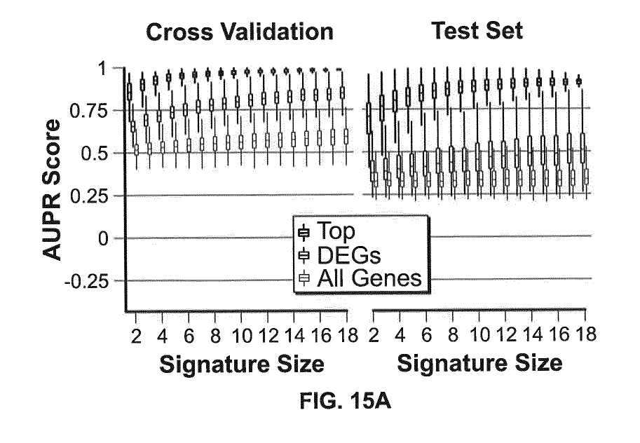

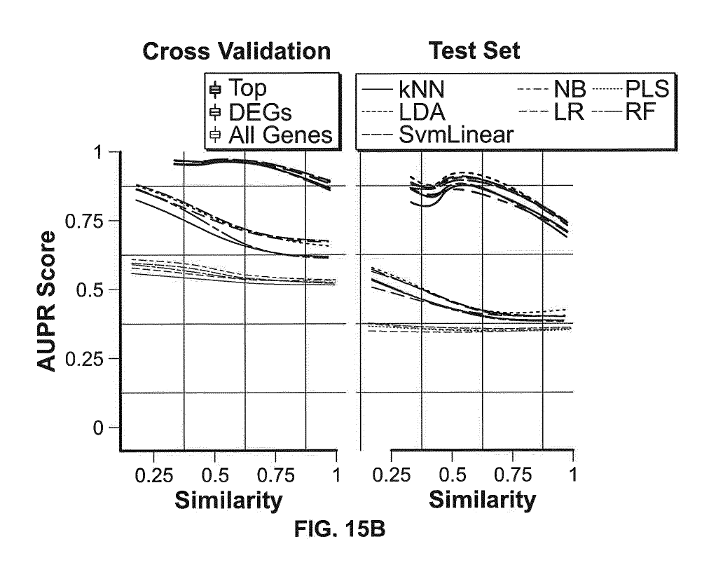

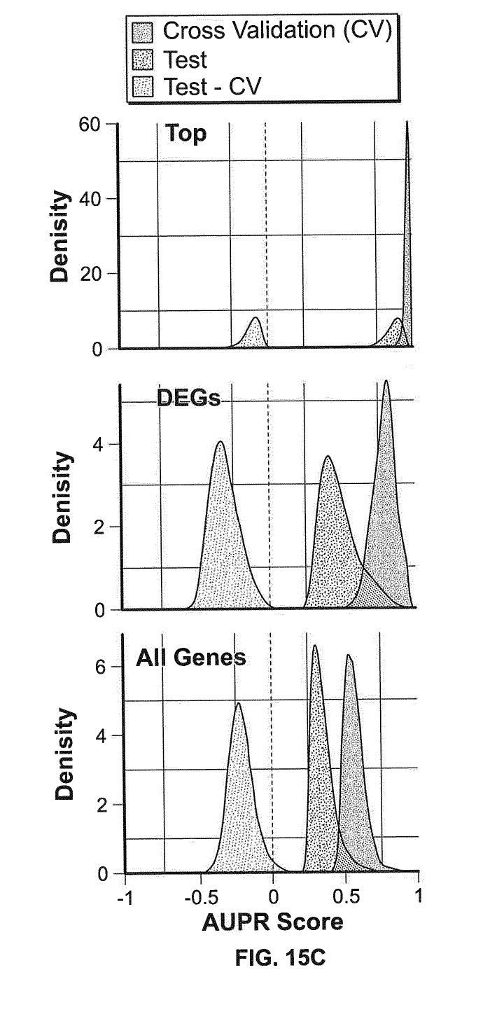

The impact of gene signature size and co-linearity level on the performance of smoking exposure status class prediction is explored using the 18 gene-based consensus signature from the top six teams' predictions. MCC and AUPR scores are calculated to evaluate the performance of all possible combinations of signatures of lengths 2 to 18 with ML-based class predictions (FIGS. 14 and 15). FIGS. 14 and 15 display results for the MCC scores (FIG. 14) and the AUPR scores (FIG. 15). In both figures, panel A depicts the score versus gene signature size for cross-validation and test data set. Features are selected from the list of (i) “Top” genes (i.e., genes selected frequently by participants as part of the signature; (ii) “DEGs”, list of differentially expressed genes; (iii) “All Genes”, all measured genes. In both figures, panel B depicts the score versus coefficient of similarity between genes in the signature. Seven different machine learning classifiers are tested: Random Forest (RF), support vector machine with linear kernel (svmLinear), partial least squares discriminant analysis (PLS), naive Bayes (NB), k-Near Neighbor (kNN), linear discriminant analysis (LDA), and logistic regression (LR). In both figures, panel C depicts distributions of the scores in CV and test set data, plus distribution of the differences for “Top” (top), “DEGs” (middle), and “All genes” (bottom) selections.

As is indicated by the data in FIGS. 14 and 15, the prediction performance increases with gene set size and gradually stabilized with longer sets, including up to 18 genes in both training (cross-validation, CV) (for CV, MCC=0.57 for size=2 and MCC=0.91 for size=18) and test sets (for test, MCC=0.42 for size=2 and MCC=0.77 for size=18) (FIG. 14A). Prediction performances reached maximum when the co-linearity level (reflected by the percentage of variance represented by the first principal component computed from the gene set expression matrix) of genes in the “Top” gene sets ranged between 50% and 60%, and then decreased with increased co-linearity (FIG. 14B). Considering that the “Top” gene sets were composed of the signature genes from different teams and were already quite diverse, combining genes that are to some extent co-linear may strengthen the prediction. Performances decreased with increased co-linearity of genes within gene sets from DEGs (FIG. 14B). In general, gene sets from “Top”, “DEG”, and “All Genes” gave the best, middle, and worst performances, respectively (FIG. 14). In addition, performances derived from CV outperformed those computed for the test set (FIG. 14). Performance metrics obtained with various ML methods showed similar patterns (FIG. 14B), and therefore, were aggregated to facilitate the visualization of results (FIG. 14A and FIG. 14C). Overall, the results indicated that blood genes from the 18 gene-based consensus signature were informative and had high predictive power for smoking exposure status when combined.

### Example 1—Discussion

The results obtained in this example study provide the predicted confidence that blood samples from subjects exposed to a candidate MRTP, or who switched to a candidate MRTP following conventional CS exposure belong to the smoke-exposed or the non-current smoke-exposed group.

The results clearly separate smokers and non-current smokers. Challenge participants successfully developed species-independent blood-based gene signature models that show very good performance for smoking exposure status prediction irrespective of human and mouse species. In the human test data set, the former smoker group, although very close to the never smoker group, remained intermediate between the smoker and never smoker groups, indicating that the expression of genes in the gene signature of a former smokers may not be completely reversed back to the expression levels of never smokers. The reversion of changes likely depends on smoking history and quitting time duration, which vary from one subject to another, also explaining the higher variability of the predictions for this group. For former smokers' blood cells, DNA methylation levels (e.g. F2RL3 gene) may depend on pack years and time since quitting.

In the mouse data set, the expression levels of the Cess group reaches the level of the Sham group, suggesting a reversion of signature gene expression changes in blood cells of mouse strain that are more genetically and experimentally homogeneous. Interestingly, this reversion occurs gradually over time, as is observed when the groups are split based on cessation time duration. This suggests that the gene signature classification approach is not only useful for binary classification, but could also be used in a more quantitative manner (e.g., magnitude of model parameters such as LDA scores or associated confidence values) to follow the magnitude and kinetics of changes that occur in blood upon product testing or withdrawal. Indeed, this is the case for the Switch and Cess groups from the verification human REX data sets, which show significant log odds decreases towards the values of the never smoker group compared with the smoker group. This observation indicates that molecular changes reflected by smoking exposure signature genes, occurs in blood cells after only 5 days switching to a candidate MRTP or quitting conventional cigarettes. These results are consistent with reductions of dose-responsive biomarkers of exposure measured after one week in a clinical “cigarette per day reduction” confinement study. For the mouse verification data sets, the difference of log odds between the 3R4F group and the prototype/candidate MRTP or Switch groups (similar level as Sham) is even more important, because it could be explained by longer (months) exposure to a candidate MRTP or pMRTP after switching, and reflected lower biological effects of MRTPs on blood cells compared with conventional CS.

The sample classification performances obtained by the top-performing teams are high even though the computational methods that are used to develop and train the blood-based smoking exposure response classification models are different. A core gene signature is identified that is highly consistent across teams, indicating that gene expression changes induced by smoke exposure are sufficiently informative and consistent to select genes that together constituted specific and robust blood markers predictive of smoking exposure status in human only or in human and mouse (species-independent signature).

Blood cell type-specific transcriptome analysis, similar to the reported DNA methylation analysis of cell-specific leukocytes from smokers and non smokers, may help to provide a better understanding of the contribution of each blood cell type to the smoking exposure response signature. Some genes may be related to specific blood cell sub-populations. Overall, these smoking exposure-associated genes, which are part of the core signature, constitute a robust set of blood markers that can be leveraged to monitor and possibly quantify the impact of new products such as candidate MRTPs compared with that of a conventional cigarette.

The study described in relation to Example 1 shows how the power of a crowd may be leveraged to evaluate computational methods and verify data in systems toxicology. In addition to complementing the classical peer review process, independent and unbiased evaluations of product risk assessment data may be used to confirm and provide confidence in scientific conclusions, and may support regulatory authorities for decision-making. While the examples described herein are mostly directed to using crowd-sourcing approaches to identify a robust gene signature for predicting an individual's smoker status, one of ordinary skill in the art will understand that the systems and methods of the present disclosure may be applied to obtain gene signatures for predicting the biological status of an individual, including smoker status, disease status, physiological state, exposure state, or any other suitable status or state of an individual that is associated with the individual's biological state.

Table 2 below includes results from a study conducted in accordance with Example 1. In particular, the results shown in Table 2 are drawn from a human smoking signature and lists a set of genes in the first column. The second column lists the number of teams or participants (out of 12) that included the corresponding gene in its signature. The third column lists the number of top 3 teams (assessed according to a test data set) that included the corresponding gene in its signature. The fourth column lists the number of top 3 teams (assessed according to a verification data set) that included the corresponding gene in its signature. The fifth column lists the mean of the values in the third and fourth columns.

TABLE 2

| | SUM | SUM Top 3 | | |
|---|---|---|---|---|
| Scoring | (out of | TEST | SUM Top 3 | MEAN |
| TEST set | 12 teams) | set | VERIF set | SET + VERIF |
| LRRN3 | 9 | 3 | 3 | 3 |
| AHRR | 9 | 3 | 3 | 3 |
| CDKN1C | 9 | 3 | 3 | 3 |
| PID1 | 8 | 3 | 3 | 3 |
| SASH1 | 7 | 3 | 3 | 3 |
| GPR15 | 7 | 3 | 3 | 3 |
| P2RY6 | 6 | 3 | 3 | 3 |
| LINC00599 | 6 | 2 | 3 | 2.5 |
| CLEC10A | 6 | 3 | 2 | 2.5 |
| SEMA6B | 5 | 2 | 3 | 2.5 |
| F2R | 5 | 2 | 2 | 2 |
| DSC2 | 5 | 1 | 0 | 0.5 |
| TLR5 | 5 | 0 | 1 | 0.5 |
| RGL1 | 4 | 1 | 2 | 1.5 |
| FSTL1 | 4 | 1 | 0 | 0.5 |
| VSIG4 | 4 | 0 | 0 | 0 |
| AK8 | 4 | 0 | 0 | 0 |
| CTTNBP2 | 3 | 2 | 2 | 2 |
| GUCY1A3 | 3 | 1 | 1 | 1 |
| GSE1 | 3 | 1 | 0 | 0.5 |
| MIR4697HG | 3 | 0 | 0 | 0 |
| PTGFRN | 3 | 0 | 0 | 0 |
| LOC200772 | 3 | 0 | 0 | 0 |
| FANK1 | 3 | 0 | 0 | 0 |
| C15orf54 | 3 | 0 | 0 | 0 |
| MARC2 | 3 | 0 | 0 | 0 |
| GPR63 | 2 | 2 | 1 | 1.5 |
| TPPP3 | 2 | 1 | 1 | 1 |
| ZNF618 | 2 | 1 | 1 | 1 |
| PTGFR | 2 | 1 | 0 | 0.5 |
| GUCY1B3 | 2 | 0 | 1 | 0.5 |
| P2RY1 | 2 | 0 | 0 | 0 |
| TMEM163 | 2 | 0 | 0 | 0 |
| ST6GALNAC1 | 2 | 0 | 0 | 0 |
| SH2D1B | 2 | 0 | 0 | 0 |
| CYP4F22 | 2 | 0 | 0 | 0 |
| PF4 | 2 | 0 | 0 | 0 |
| FUCA1 | 2 | 0 | 0 | 0 |
| MB21D2 | 2 | 0 | 0 | 0 |
| NLK | 2 | 0 | 0 | 0 |
| B3GALT2 | 2 | 0 | 0 | 0 |
| ASGR2 | 2 | 0 | 0 | 0 |
| NR4A1 | 2 | 0 | 0 | 0 |
| RTN1 | 1 | 1 | 1 | 1 |
| MAFB | 1 | 1 | 1 | 1 |
| ARHGEF10L | 1 | 1 | 1 | 1 |
| CLDN23 | 1 | 1 | 1 | 1 |
| TGFBI | 1 | 1 | 1 | 1 |
| LOC284837 | 1 | 1 | 1 | 1 |
| SYCE1L | 1 | 1 | 1 | 1 |
| SEZ6L | 1 | 1 | 1 | 1 |
| KLF4 | 1 | 1 | 1 | 1 |
| NOD1 | 1 | 1 | 1 | 1 |
| FAM225A | 1 | 1 | 1 | 1 |
| CRACR2B | 1 | 1 | 0 | 0.5 |

In some embodiments, the gene signature used for determining a smoking exposure response status includes the genes listed in Table 2 corresponding to genes appearing in at least two of the top three-performing gene signatures. When assessed according to the test data set (e.g., shown in the third column of Table 2), this includes LRRN3, AHRR, CDKN1C, PID1, SASH1, GPR15, P2RY6, LINC00599, CLEC10A, SEMA6B, F2R, CTTNBP2, and GPR63. When assessed according to the verification data set (e.g., shown in the fourth column of Table 2), this includes LRRN3, AHRR, CDKN1C, PID1, SASH1, GPR15, P2RY6, LINC00599, CLEC10A, SEMA6B, F2R, RGL1, and CTTNBP2. When assessed according to the mean between the test and verification data sets (e.g., shown in the fifth column of Table 2), this includes LRRN3, AHRR, CDKN1C, PID1, SASH1, GPR15, P2RY6, LINC00599, CLEC10A, SEMA6B, F2R, and CTTNBP2.

In some embodiments, the gene signature used for determining a smoking exposure response status includes the genes listed in Table 2 corresponding to genes appearing in at least M of the twelve candidate gene signatures, where M is 1, 2, 3, 4, 5, 6, 7, 8, or 9. For example, when M is 9, the gene signature includes those genes with a value of at least 9 in the second column, namely: LRRN3, AHRR, and CDKN1C. As another example, when M is 8, the gene signature includes those genes with a value of at least 8 in the second column, namely: LRRN3, AHRR, CDKN1C, and PID1. As another example, when M is 7, the gene signature includes those genes with a value of at least 7 in the second column, namely: LRRN3, AHRR, CDKN1C, PID1, SASH1, and GPR15. As another example, when M is 6, the gene signature includes those genes with a value of at least 6 in the second column, namely: LRRN3, AHRR, CDKN1C, PID1, SASH1, GPR15, P2RY6, LINC00599, and CLEC10A. As another example, when M is 5, the gene signature includes those genes with a value of at least 5 in the second column, namely: LRRN3, AHRR, CDKN1C, PID1, SASH1, GPR15, P2RY6, LINC00599, CLEC10A, SEMA6B, F2R, DSC2, and TLR5. As another example, when M is 4, the gene signature includes those genes with a value of at least 4 in the second column, namely: LRRN3, AHRR, CDKN1C, PID1, SASH1, GPR15, P2RY6, LINC00599, CLEC10A, SEMA6B, F2R, DSC2, TLR5, RGL1, FSTL1, VSIG4, and AK8. As another example, when M is 3, the gene signature includes those genes with a value of at least 3 in the second column, namely: LRRN3, AHRR, CDKN1C, PID1, SASH1, GPR15, P2RY6, LINC00599, CLEC10A, SEMA6B, F2R, DSC2, TLR5, RGL1, FSTL1, VSIG4, AK8, CTTNBP2, GUCY1A3, GSE1, MIR4697HG, PTGFRN, LOC200772, FANK1, C15orf54, and MARC2. As another example, when M is 2, the gene signature includes those genes with a value of at least 2 in the second column, namely: LRRN3, AHRR, CDKN1C, PID1, SASH1, GPR15, P2RY6, LINC00599, CLEC10A, SEMA6B, F2R, DSC2, TLR5, RGL1, FSTL1, VSIG4, AK8, CTTNBP2, GUCY1A3, GSE1, MIR4697HG, PTGFRN, LOC200772, FANK1, C15orf54, MARC2, GPR63, TPPP3, ZNF618, PTGFR, GUCY1B3, P2RY1, TMEM163, ST6GALNAC1, SH2D1B, CYP4F22, PF4, FUCA1, MB21D2, NLK, B3GALT2, ASGR2, and NR4A1. As another example, when M is 1, the gene signature includes all the genes listed in Table 2 above.

Table 3 below includes results from a study conducted in accordance with Example 1. In particular, the results shown in Table 2 are drawn from a species-independent smoking signature and lists a set of genes in the first column. The second column lists the number of teams or participants (out of 12) that included the corresponding gene in its signature. The third column lists the number of top 3 teams (assessed according to a test data set) that included the corresponding gene in its signature. The fourth column lists the number of top 3 teams (assessed according to a verification data set) that included the corresponding gene in its signature. The fifth column lists the mean of the values in the third and fourth columns.

TABLE 3

| | SUM (out of | SUM Top 3 | SUM Top 3 | |
|---|---|---|---|---|
| Scoring | 12 | TEST | VERIF | MEAN |
| TEST set | teams) | set | set | SET + VERIF |
| AHRR | 5 | 3 | 3 | 3 |
| P2RY6 | 4 | 3 | 3 | 3 |
| COX6B2 | 2 | 2 | 2 | 2 |
| DSC2 | 2 | 2 | 2 | 2 |
| KLRG1 | 3 | 2 | 2 | 2 |
| LRRN3 | 3 | 2 | 2 | 2 |
| SASH1 | 2 | 2 | 2 | 2 |
| TBX21 | 2 | 2 | 2 | 2 |
| ADORA3 | 1 | 1 | 1 | 1 |
| AF529169 | 1 | 1 | 1 | 1 |
| AKAP5 | 1 | 1 | 1 | 1 |
| ASGR2 | 1 | 1 | 1 | 1 |
| B3GALT2 | 1 | 1 | 1 | 1 |
| BCL3 | 1 | 1 | 1 | 1 |
| BIRC2 | 1 | 1 | 1 | 1 |
| CCR4 | 1 | 1 | 1 | 1 |
| CDKN1C | 1 | 1 | 1 | 1 |
| CLEC10A | 1 | 1 | 1 | 1 |
| CLEC5A | 1 | 1 | 1 | 1 |
| CNNM1 | 1 | 1 | 1 | 1 |
| COL6A3 | 1 | 1 | 1 | 1 |
| COX6C | 1 | 1 | 1 | 1 |
| CRACR2B | 1 | 1 | 1 | 1 |
| CTNNAL1 | 1 | 1 | 1 | 1 |
| CTTNBP2 | 2 | 1 | 1 | 1 |
| DCAF8 | 1 | 1 | 1 | 1 |
| EIF5A2 | 1 | 1 | 1 | 1 |
| ELOVL7 | 1 | 1 | 1 | 1 |
| ENDOU | 1 | 1 | 1 | 1 |
| ERI1 | 1 | 1 | 1 | 1 |
| ESAM | 1 | 1 | 1 | 1 |
| EVA1B | 1 | 1 | 1 | 1 |
| F2R | 2 | 1 | 1 | 1 |
| FANK1 | 1 | 1 | 1 | 1 |
| FKRP | 1 | 1 | 1 | 1 |
| FSTL1 | 1 | 1 | 1 | 1 |
| GGT7 | 1 | 1 | 1 | 1 |
| GLCCI1 | 1 | 1 | 1 | 1 |
| GNAZ | 1 | 1 | 1 | 1 |
| GNPDA2 | 1 | 1 | 1 | 1 |
| GP1BA | 1 | 1 | 1 | 1 |
| GPR63 | 1 | 1 | 1 | 1 |
| GSE1 | 1 | 1 | 1 | 1 |
| GUCY1B3 | 2 | 1 | 1 | 1 |
| HES1 | 1 | 1 | 1 | 1 |
| HPGD | 1 | 1 | 1 | 1 |
| HSPB6 | 1 | 1 | 1 | 1 |
| IRF7 | 1 | 1 | 1 | 1 |
| JARID2 | 1 | 1 | 1 | 1 |
| KCNQ1OT1 | 1 | 1 | 1 | 1 |
| KISS1R | 1 | 1 | 1 | 1 |
| LIMS1 | 1 | 1 | 1 | 1 |
| LRRK1 | 1 | 1 | 1 | 1 |
| LTBP1 | 1 | 1 | 1 | 1 |
| MBTD1 | 1 | 1 | 1 | 1 |
| MCEMP1 | 1 | 1 | 1 | 1 |
| MKNK1 | 1 | 1 | 1 | 1 |
| MPP2 | 1 | 1 | 1 | 1 |
| MRAS | 1 | 1 | 1 | 1 |
| MT2 | 2 | 1 | 1 | 1 |
| NDUFA3 | 1 | 1 | 1 | 1 |
| NGFRAP1 | 2 | 1 | 1 | 1 |
| NR4A1 | 1 | 1 | 1 | 1 |
| PF4 | 1 | 1 | 1 | 1 |
| PGRMC1 | 1 | 1 | 1 | 1 |
| PHACTR3 | 1 | 1 | 1 | 1 |
| PID1 | 1 | 1 | 1 | 1 |
| PTGFR | 1 | 1 | 1 | 1 |
| R3HDM4 | 1 | 1 | 1 | 1 |
| RBM43 | 1 | 1 | 1 | 1 |
| REEP6 | 2 | 1 | 1 | 1 |
| REXO2 | 1 | 1 | 1 | 1 |
| RUNDC3A | 1 | 1 | 1 | 1 |
| SAMD11 | 1 | 1 | 1 | 1 |
| SDR16C5 | 1 | 1 | 1 | 1 |
| SIAH1A | 1 | 1 | 1 | 1 |
| SLPI | 1 | 1 | 1 | 1 |
| SPINK2 | 1 | 1 | 1 | 1 |
| STAR | 1 | 1 | 1 | 1 |
| SYTL4 | 1 | 1 | 1 | 1 |
| TCEAL8 | 1 | 1 | 1 | 1 |
| TLR2 | 1 | 1 | 1 | 1 |
| TMEM163 | 1 | 1 | 1 | 1 |
| TRIB3 | 1 | 1 | 1 | 1 |
| UBE2B | 1 | 1 | 1 | 1 |
| VCAN | 1 | 1 | 1 | 1 |
| VSIG4 | 1 | 1 | 1 | 1 |
| WDFY1 | 1 | 1 | 1 | 1 |
| ZFP704 | 1 | 1 | 1 | 1 |

In some embodiments, the gene signature used for determining a smoking exposure response status includes the genes listed in Table 3 corresponding to genes appearing in at least two of the top three-performing gene signatures. As is shown in Table 3, regardless of whether this is assessed according to the test data set (e.g., shown in the third column of Table 3), the verification data set (e.g., shown in the fourth column of Table 3), or the mean between the test and verification data sets (e.g., shown in the fifth column of Table 3), this includes AHRR, P2RY6, COX6B2, DSC2, KLRG1, LRRN3, SASH1, and TBX21.

In some embodiments, the gene signature used for determining a smoking exposure response status includes the genes listed in Table 3 corresponding to genes appearing in at least M of the 12 submitted gene signatures, where M is 1, 2, 3, 4, or 5. For example, when M is 5, the gene signature includes those genes with a value of at least 5 in the second column, namely: AHRR. As another example, when M is 4, the gene signature includes those genes with a value of at least 4 in the second column, namely: AHRR and P2RY6. As another example, when M is 3, the gene signature includes those genes with a value of at least 3 in the second column, namely: AHRR, P2RY6, KLRG1, and LRRN3. As another example, when M is 2, the gene signature includes those genes with a value of at least 2 in the second column, namely: AHRR, P2RY6, KLRG1, LRRN3, COX6B2, DSC2, SASH1, TBX21, CTTNBP2, F2R, GUCY1B3, MT2, NGFRAP1, and REEP6. As another example, when M is 1, the gene signature includes all the genes listed in Table 3 above.

In some embodiments, the gene signatures described herein are restricted to have a maximum number of genes, such as 10, 11, 12, 13, 14, 15, 20, 25, 30, 35, 40, or any other suitable number less than the number of genes in the whole genome. The gene signatures described here are restricted to a relatively small number of genes compared to the whole genome. A longer gene signature may perform worse than a shorter gene signature, if the longer gene signature is over-fitted to the training data set. In this case, the longer gene signature may describe random error or noise in the training data set. When being used to predict classes in the test data set, a shorter gene signature may outperform the over-fitted longer gene signature. Any of the gene signatures described herein, including the gene signatures described in relation to Tables 2 and 3, may be restricted to have a particular maximum number of genes.

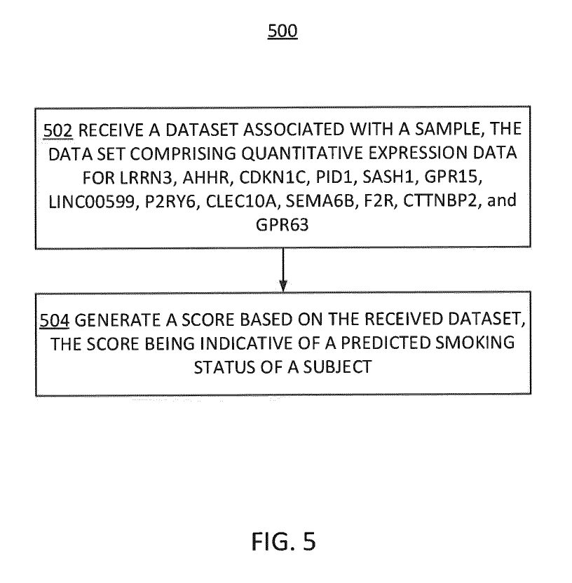

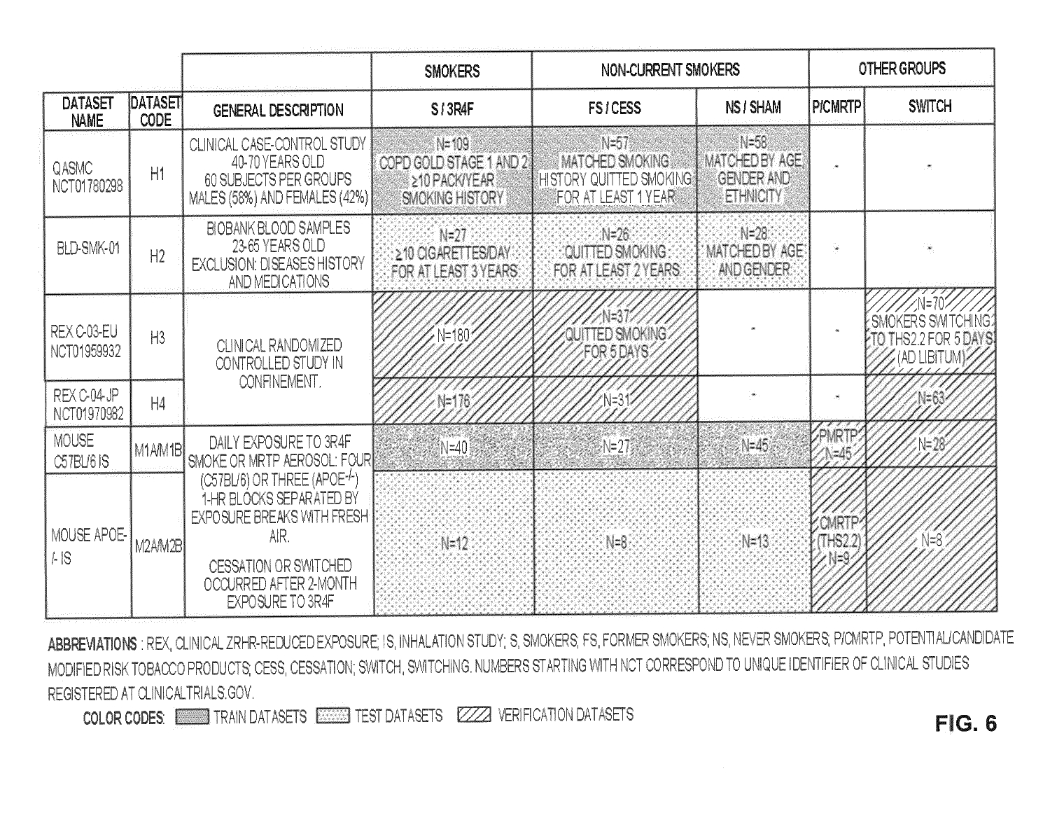

FIG. 5 is a flowchart of a process 500 for assessing a sample obtained from a subject, according to an illustrative embodiment of the disclosure. The process 500 includes the steps of receiving a data set associated with a sample, the data set comprising quantitative expression data for LRRN3, AHHR, CDKN1C, PID1, SASH1, GPR15, LINC00599, P2RY6, CLEC10A, SEMA6B, F2R, CTTNBP2, and GPR63 (step 502), and generating a score based on the received data set, where the score is indicative of a predicted smoking status of a subject (step 504). In some embodiments, the data set received at step 502 further comprises quantitative expression data for any number of the following: DSC2, TLR5, RGL1, FSTL1, VSIG4, AK8, GUCY1A3, GSE1, MIR4697HG, PTGFRN, LOC200772, FANK1, C15orf54, MARC2, TPPP3, ZNF618, PTGFR, P2RY1, TMEM163, ST6GALNAC1, SH2D1B, CYP4F22, PF4, FUCA1, MB21D2, NLK, B3GALT2, ASGR2, NR4A1, and GUCY1B3. In some embodiments, the data set received at step 502 further comprises quantitative expression data for any of the gene signatures described in relation to Tables 2 and 3 above, or any other the gene signatures described herein.

The score generated at step 504 is a result of a classification scheme applied to the data set, wherein the classification scheme is determined based on the quantitative expression data in the data set. In particular, in the examples described herein, the classifier that was trained using a machine learning technique may be applied to the data set received at 502 to determine a predicted classification for the individual.

The gene signatures described herein may be used in a computer-implemented method for assessing a sample obtained from a subject. In particular, a data set associated with the sample may be obtained, and the data set may include quantitative expression data for LRRN3, AHHR, CDKN1C, PID1, SASH1, GPR15, LINC00599, P2RY6, CLEC10A, SEMA6B, F2R, CTTNBP2, and GPR63 for the core gene signature. In general, any of the gene signatures described in relation to Tables 2 and 3 may be used as the core gene signature. The core gene signature includes a number of genes that is less than the number of genes in the entire genome, and includes a set of genes that, when considered together as a whole, are informative for predicting a biological state such as smoking status. A score may be generated based on the gene signature in the received data set, where the score is indicative of a predicted smoking status of the subject. In particular, the score may be based on a classifier that was built using the crowd-sourcing approach described herein. The data set may further comprise quantitative expression data for any suitable combination of the additional markers DSC2, TLR5, RGL1, FSTL1, VSIG4, AK8, GUCY1A3, GSE1, MIR4697HG, PTGFRN, LOC200772, FANK1, C15orf54, MARC2, TPPP3, ZNF618, PTGFR, P2RY1, TMEM163, ST6GALNAC1, SH2D1B, CYP4F22, PF4, FUCA1, MB21D2, NLK, B3GALT2, ASGR2, NR4A1, and GUCY1B3, which may be included in an extended gene signature. The data set may further comprise quantitative expression data for any of the gene signatures described in relation to Tables 2 and 3 above.

In some embodiments, the data set includes any number of any subset of the set of markers LRRN3, AHHR, CDKN1C, PID1, SASH1, GPR15, LINC00599, P2RY6, CLEC10A, SEMA6B, F2R, CTTNBP2, and GPR63. The subset may include less than all of these identified genes. One or more criteria may be applied to the markers to be included in a signature, such as including at least three (or any other suitable number, such as 4, 5, 6, 7, 8, 9, 10, 11, or 12) of markers in a core set: LRRN3, AHHR, CDKN1C, PID1, SASH1, GPR15, LINC00599, P2RY6, CLEC10A, SEMA6B, F2R, CTTNBP2, and GPR63, and at least two (or any other suitable number, such as 2, 3, 4, 5, 6, 7, 8, 9, 10, 11, or 12) of any of the markers in the gene signatures described in relation to Tables 2 or 3. As described above, in some embodiments, the signature is limited to a number of genes that is less than the number of genes in the entire genome and may be limited to a maximum number of genes, such as 10, 11, 12, 13, 14, 15, 20, 25, 30, 35, 40, or any other suitable number less than the number of genes in the whole genome. In general, any signature using a combination of these markers may be used for predicting the biological status of a subject, such as smoking status, without departing from the scope of the present disclosure.

In some embodiments, the genes in the signatures described herein are used in assembling a kit for predicting smoker status of an individual. In particular, the kit includes a set of reagents that detects expression levels of the genes in the gene signature in a test sample, and instructions for using the kit for predicting smoker status in the individual. The kit may be used to assess an effect of cessation or an alternative to a smoking product on an individual, such as an HTP.

FIG. 2 is a block diagram of a computing device for performing any of the processes described herein, such as the processes described in relation to FIGS. 1 and 2, or for storing the core gene signature, extended gene signature, or any other gene signature described herein. In particular, the gene signature that is stored on a computer readable medium includes expression data for LRRN3, AHHR, CDKN1C, PID1, SASH1, GPR15, LINC00599, P2RY6, CLEC10A, SEMA6B, F2R, CTTNBP2, and GPR63. In another example, the computer readable medium includes a gene signature that includes expression data for at least 4, 5, 6, 7, 8, 9, 10, 11, or 12 markers selected from the group consisting of: LRRN3, AHHR, CDKN1C, PID1, SASH1, GPR15, LINC00599, P2RY6, CLEC10A, SEMA6B, F2R, CTTNBP2, and GPR63. In another example, the computer readable medium includes data related to any of the gene signatures or set of markers described herein.

In certain implementations, a component and a database may be implemented across several computing devices 200. The computing device 200 comprises at least one communications interface unit, an input/output controller 210, system memory, and one or more data storage devices. The system memory includes at least one random access memory (RAM 202) and at least one read-only memory (ROM 204). All of these elements are in communication with a central processing unit (CPU 206) to facilitate the operation of the computing device 200. The computing device 200 may be configured in many different ways. For example, the computing device 200 may be a conventional standalone computer or alternatively, the functions of computing device 200 may be distributed across multiple computer systems and architectures. The computing device 200 may be configured to perform some or all of modeling, scoring and aggregating operations. In FIG. 2, the computing device 200 is linked, via network or local network, to other servers or systems.

The computing device 200 may be configured in a distributed architecture, wherein databases and processors are housed in separate units or locations. Some such units perform primary processing functions and contain at a minimum a general controller or a processor and a system memory. In such an aspect, each of these units is attached via the communications interface unit 208 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 206 comprises a processor, such as one or more conventional microprocessors and one or more supplementary co-processors such as math co-processors for offloading workload from the CPU 206. The CPU 206 is in communication with the communications interface unit 208 and the input/output controller 210, through which the CPU 206 communicates with other devices such as other servers, user terminals, or devices. The communications interface unit 208 and the input/output controller 210 may include multiple communication channels for simultaneous communication with, for example, other processors, servers or client terminals. Devices in communication with each other need not be continually transmitting to each other. On the contrary, such devices need only transmit to each other as necessary, may actually refrain from exchanging data most of the time, and may require several steps to be performed to establish a communication link between the devices.

The CPU 206 is also in communication with the data storage device. The data storage device may comprise an appropriate combination of magnetic, optical or semiconductor memory, and may include, for example, RAM 202, ROM 204, flash drive, an optical disc such as a compact disc or a hard disk or drive. The CPU 206 and the data storage device each may be, for example, located entirely within a single computer or other computing device; or connected to each other by a communication medium, such as a USB port, serial port cable, a coaxial cable, an Ethernet type cable, a telephone line, a radio frequency transceiver or other similar wireless or wired medium or combination of the foregoing. For example, the CPU 206 may be connected to the data storage device via the communications interface unit 208. The CPU 206 may be configured to perform one or more particular processing functions.

The data storage device may store, for example, (i) an operating system 212 for the computing device 200; (ii) one or more applications 214 (e.g., computer program code or a computer program product) adapted to direct the CPU 206 in accordance with the systems and methods described here, and particularly in accordance with the processes described in detail with regard to the CPU 206; or (iii) database(s) 216 adapted to store information that may be utilized to store information required by the program. In some aspects, the database(s) includes a database storing experimental data, and published literature models.

The operating system 212 and applications 214 may be stored, for example, in a compressed, an uncompiled and an encrypted format, and may include computer program code. The instructions of the program may be read into a main memory of the processor from a computer-readable medium other than the data storage device, such as from the ROM 204 or from the RAM 202. While execution of sequences of instructions in the program causes the CPU 206 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions as described herein. The program also may include program elements such as an operating system 212, a database management system and “device drivers” that allow the processor to interface with computer peripheral devices (e.g., a video display, a keyboard, a computer mouse, etc.) via the input/output controller 210.

The term “computer-readable medium” as used herein refers to any non-transitory medium that provides or participates in providing instructions to the processor of the computing device 200 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer may read.

Various forms of computer readable media may be involved in carrying one or more sequences of one or more instructions to the CPU 206 (or any other processor of a device described herein) for execution. For example, the instructions may initially be borne on a magnetic disk of a remote computer (not shown). The remote computer may load the instructions into its dynamic memory and send the instructions over an Ethernet connection, cable line, or even telephone line using a modem. A communications device local to a computing device 200 (e.g., a server) may receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

Each reference that is referred to herein is hereby incorporated by reference in its respective entirety.

While implementations of the disclosure have been particularly shown and described with reference to specific examples, it should be understood by those skilled in the art that various changes in form and detail may be made therein without departing from the scope of the disclosure as defined by the appended claims. The scope of the disclosure is thus indicated by the appended claims and all changes which come within the meaning and range of equivalency of the claims are therefore intended to be embraced.

1. A computer-implemented method for assessing a sample obtained from a subject, comprising:
receiving, by a computer system including at least one hardware processor a data set associated with the sample, the data set comprising quantitative expression data for a set of genes less than a whole genome, the set of genes comprising AHHR, CDKN1C, LRRN3, PID1, GPR15, SASH1, CLEC10A, LINC00599, P2RY6, DSC2, F2R, SEMA6B, and TLR5; and
generating, by the at least one hardware processor, a score based on the quantitative expression data for the set of genes in the received data set, wherein the score is based on fewer than 40 genes and is indicative of a predicted smoking status of the subject.

2. The computer-implemented method of claim 1, wherein the set of genes further comprises AK8, FSTL1, RGL1, and VSIG4.

3. The computer-implemented method of claim 1, wherein the set of genes further comprises C15orf54, CTTNBP2, RANK1, GSE1, GUCY1A3, LOC200772, MARC2, MIR4697HG, and PTGFRN.

4. The computer-implemented method of claim 1, wherein the score is a result of a classification scheme applied to the data set, wherein the classification scheme is determined based on the quantitative expression data in the data set.

5. The computer-implemented method of claim 1, further comprising computing a fold-change value for each of AHHR, CDKN1C, LRRN3, PID1, GPR15, SASH1, CLEC10A, LINC00599, P2RY6, DSC2, F2R, SEMA6B, and TLR5.

6. The computer-implemented method of claim 5, further comprising determining that each fold-change value satisfies at least one criterion that requires that each respective computed fold-change value exceeds a predetermined threshold for at least two independent population data sets.

7. The computer-implemented method of claim 1, wherein the set of genes consists of AHHR, CDKN1C, LRRN3, PID1, GPR15, SASH1, CLEC10A, LINC00599, P2RY6, DSC2, F2R, SEMA6B, and TLR5.

8. A non-transitory computer-readable medium having instructions stored thereon that, when executed by at least one computing device, cause the at least one computing device to perform operations comprising the method of claim 1.

9. A kit for predicting smoker status of an individual, comprising:
a set of reagents that detects expression levels of the genes in a gene signature having fewer than 40 genes, the gene signature comprising AHHR, CDKN1C, LRRN3, PID1, GPR15, SASH1, CLEC10A, LINC00599, P2RY6, DSC2, F2R, SEMA6B, and TLR5 in a test sample; and
instructions for using said kit for predicting smoker status in the individual.

10. The kit of claim 9, wherein the kit is used for assessing an effect of an alternative to a smoking product on an individual.

11. The kit of claim 10, wherein the alternative to the smoking product is a heated tobacco product.

12. The kit of claim 9, wherein the effect of the alternative on the individual is to classify the individual as a non-smoker.

13. The kit of claim 9, wherein the gene signature further comprises AK8, FSTL1, RGL1, and VSIG4.

14. The kit of claim 9, wherein the gene signature further comprises C15orf54, CTTNBP2, RANK1, GSE1, GUCY1A3, LOC200772, MARC2, MIR4697HG, and PTGFRN.

15. A computer-implemented method for assessing a sample obtained from a subject, comprising:
receiving, by a computer system including at least one hardware processor, a data set associated with the sample, the data set comprising quantitative expression data for a set of genes less than a whole genome, the set of genes comprising LRRN3, AHHR, CDKN1C, PID1, SASH1, GPR15, LINC00599, P2RY6, CLEC10A, SEMA6B, F2R, CTTNBP2, and GPR63; and
generating, by the at least one hardware processor, a score based on the quantitative expression data for the set of genes in the received data set, wherein the score is based on fewer than 40 genes and is indicative of a predicted smoking status of the subject.

16. The computer-implemented method of claim 15, wherein the score is a result of a classification scheme applied to the data set, wherein the classification scheme is determined based on the quantitative expression data in the data set.

17. The computer-implemented method of claim 15, further comprising computing a fold-change value for each of LRRN3, AHHR, CDKN1C, PID1, SASH1, GPR15, LINC00599, P2RY6, CLEC10A, SEMA6B, F2R, CTTNBP2, and GPR63.

18. The computer-implemented method of claim 17, further comprising determining that each fold-change value satisfies at least one criterion that requires that each respective computed fold-change value exceeds a predetermined threshold for at least two independent population data sets.

19. The computer-implemented method of claim 15, wherein the set of genes consists of LRRN3, AHHR, CDKN1C, PID1, SASH1, GPR15, LINC00599, P2RY6, CLEC10A, SEMA6B, F2R, CTTNBP2, and GPR63.

20. A non-transitory computer-readable medium having instructions stored thereon that, when executed by at least one computing device, cause the at least one computing device to perform operations comprising the method of claim 15.

21. A kit for predicting smoker status of an individual, comprising:
a set of reagents that detects expression levels of the genes in a gene signature having fewer than 40 genes, the gene signature comprising LRRN3, AHHR, CDKN1C, PID1, SASH1, GPR15, LINC00599, P2RY6, CLEC10A, SEMA6B, F2R, CTTNBP2, and GPR63 in a test sample; and
instructions for using said kit for predicting smoker status in the individual.

22. The kit of claim 21, wherein the kit is used for assessing an effect of an alternative to a smoking product on an individual.

23. The kit of claim 22, wherein the alternative to the smoking product is a heated tobacco product.

24. The kit of claim 21, wherein the effect of the alternative on the individual is to classify the individual as a non-smoker.

25-45. (canceled)

46. A computer-implemented method for assessing a sample obtained from a subject, comprising:
receiving, by a computer system including at least one hardware processor, a data set associated with the sample, the data set comprising quantitative expression data for a set of genes less than a whole genome, the set of genes comprising AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618; and
generating, by the at least one hardware processor, a score based on the received data set, wherein the score is indicative of a predicted smoking status of the subject.

47. The computer-implemented method of claim 46, wherein the score is a result of a classification scheme applied to the data set, wherein the classification scheme is determined based on the quantitative expression data in the data set.

48. The computer-implemented method of claim 46, further comprising computing a fold-change value for each of AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618.

49. The computer-implemented method of claim 48, further comprising determining that each fold-change value satisfies at least one criterion that requires that each respective computed fold-change value exceeds a predetermined threshold for at least two independent population data sets.

50. The computer-implemented method of claim 46, wherein the set of genes consists of AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618.

51. A non-transitory computer-readable medium having instructions stored thereon that, when executed by at least one computing device, causes the at least one computing device to perform operations comprising the method of claim 46.

52. A kit for predicting smoker status of an individual, comprising:
a set of reagents that detects expression levels of the genes in a gene signature in a test sample, the gene signature comprising AHHR, CDKN1C, LRRN3, PID1, GPR15, SASH1, CLEC10A, LINC00599, P2RY6, DSC2, F2R, SEMA6B, TLR5, AK8, FSTL1, RGL1, VSIG4, C15orf54, CTTNBP2, RANK1, GSE1, GUCY1A3, LOC200772, MARC2, MIR4697HG, PTGFRN, ASGR2, B3GALT2, CYP4F22, FUCA1, GPR63, GUCY1B3, MB21D2, NLK, NR4A1, P2RY1, PF4, PTGFR, SH2D1B, ST6GALNAC1, TMEM163, TPPP3, and ZNF618; and
instructions for using said kit for predicting smoker status in the individual.

53. The kit of claim 52, wherein the kit is used for assessing an effect of an alternative to a smoking product on an individual.

54. The kit of claim 53, wherein the alternative to the smoking product is a heated tobacco product.

55. The kit of claim 52, wherein the effect of the alternative on the individual is to classify the individual as a non-smoker.

56. A computer-implemented method for assessing a sample obtained from a subject, comprising:
receiving, by a computer system including at least one hardware processor, a data set associated with the sample, the data set comprising quantitative expression data for a set of genes less than a whole genome, the set of genes comprising AHHR, P2RY6, KLRG1, LRRN3, COX6B2, CTTNBP2, DSC2, F2R, GUCY1B3, MT2, NGFRAP1, REEP6, SASH1, and TBX21; and
generating, by the at least one hardware processor, a score based on the quantitative expression data for the set of genes in the received data set, wherein the score is based on fewer than 40 genes and is indicative of a predicted smoking status of the subject.

57. The computer-implemented method of claim 56, wherein the score is a result of a classification scheme applied to the data set, wherein the classification scheme is determined based on the quantitative expression data in the data set.

58. The computer-implemented method of claim 56, further comprising computing a fold-change value for each of AHHR, P2RY6, KLRG1, LRRN3, COX6B2, CTTNBP2, DSC2, F2R, GUCY1B3, MT2, NGFRAP1, REEP6, SASH1, and TBX21.

59. The computer-implemented method of claim 58, further comprising determining that each fold-change value satisfies at least one criterion that requires that each respective computed fold-change value exceeds a predetermined threshold for at least two independent population data sets.

60. The computer-implemented method of claim 56, wherein the set of genes consists of AHHR, P2RY6, KLRG1, LRRN3, COX6B2, CTTNBP2, DSC2, F2R, GUCY1B3, MT2, NGFRAP1, REEP6, SASH1, and TBX21.

61. A non-transitory computer-readable medium having instructions stored thereon that, when executed by at least one computing device, causes the at least one computing device to perform operations comprising the method of claim 56.

62. A kit for predicting smoker status of an individual, comprising:
a set of reagents that detects expression levels of the genes in a gene signature in a test sample, the gene signature comprising AHHR, P2RY6, KLRG1, LRRN3, COX6B2, CTTNBP2, DSC2, F2R, GUCY1B3, MT2, NGFRAP1, REEP6, SASH1, and TBX21, the gene signature comprising fewer than 40 genes; and
instructions for using said kit for predicting smoker status in the individual.

63. The kit of claim 62, wherein the kit is used for assessing an effect of an alternative to a smoking product on an individual.

64. The kit of claim 63, wherein the alternative to the smoking product is a heated tobacco product.

65. The kit of claim 63, wherein the effect of the alternative on the individual is to classify the individual as a non-smoker.
